(19) United States
(12) Patent Application Publication
(10) Pub. No.: US 2017/0219590 A1
(43) Pub. Date: Aug. 3, 2017

(54) HEPATOCELLULAR CARCINOMA MARKER

(71) Applicant: National Institute of Advanced Industrial Science and Technology, Chiyoda-ku, Tokyo (JP)

(72) Inventors: Atsushi KUNO, Tsukuba-shi, Ibaraki (JP); Takashi SATO, Tsukuba-shi, Ibaraki (JP); Atsushi MATSUDA, Tsukuba-shi, Ibaraki (JP); Hisashi NARIMATSU, Tsukuba-shi, Ibaraki (JP); Hiroyuki KAJI, Tsukuba-shi, Ibaraki (JP); Akira TOGAYACHI, Tsukuba-shi, Ibaraki (JP); Ken SHIRABE, Maebashi-shi, Gunma (JP); Yoshihiko MAEHARA, Higashi-ku, Fukuoka (JP)

(73) Assignee: National Institute of Advanced Industrial Science and Technology, Chiyoda-ku, Tokyo (JP)

(21) Appl. No.: 15/327,154

(22) PCT Filed: Jul. 22, 2015
(86) PCT No.: PCT/JP2015/070894

§ 371 Date: Apr. 7, 2017

(30) Foreign Application Priority Data
Jul. 22, 2014 (JP) 2014-149270

(51) Int. Cl. G01N 33/574

(52) U.S. Cl. CPC G01N 33/57438; G01N 33/57492; G01N 2333/71; G01N 2333/96472; G01N 2333/70596

(57) ABSTRACT

The problem addressed by the present invention is to provide a marker for detecting hepatocellular carcinoma, wherein the hepatocellular carcinoma marker comprises a glycoprotein that first becomes present in the liver with the occurrence of cancer, without depending on changes in the state of the liver. The present invention provides a hepatocellular carcinoma marker comprising an NPA lectin-binding glycoprotein having an NPA lectin-binding glycan epitope that has at least one of the following properties (1) to (5): (1) the glycan epitope does not include core fucose (fucose α1→6 glycan); (2) the glycan epitope comprises a complex-type glycan having three (four or fewer) mannoses; (3) the glycan epitope does not include a high-mannose-type glycan having five or more mannoses; (4) the glycan epitope comprises a complex-type glycan that does not depend on the property of binding to LCA lectin; and (5) the glycan epitope comprises a complex-type glycan that does not depend on the property of binding to ConA lectin. By detecting the hepatocellular carcinoma marker of the present invention in a test sample, it is possible to determine the presence of hepatocellular carcinoma or the level of progression or malignancy of carcinoma.

### TECHNICAL FIELD

The present invention relates to a novel hepatocellular carcinoma marker for accurately and conveniently diagnosing hepatocellular carcinoma, and a method of testing for hepatocellular carcinoma using said marker. More specifically, the invention relates to a testing method for early detection of hepatocellular carcinoma and for providing a prognosis for patients suffering from carcinoma, and further relates to a testing reagent kit for use in testing. Specifically, the invention involves identifying a glycoprotein that is not expressed in the non-cancerous regions of hepatic tissue, but is specifically expressed in hepatocellular carcinoma or in the interstitial regions in the periphery of cancer cells (TME) in the cancerous regions, and provides a hepatocellular carcinoma marker comprising this glycoprotein. Additionally, the invention offers a testing method for hepatocellular carcinoma using a lectin that binds to said glycoprotein, and a kit therefor.

### BACKGROUND ART

In Japan, cancer (malignant neoplasm) continues to increase as a major cause of death, and has been the number one cause of death since 1981. In the year 2011, it claimed 28.5% of all deaths, significantly widening its lead in number of deaths relative to other diseases such as heart disease, pneumonia and brain disease. In other words, about one in every 3.5 deaths was caused by cancer.

Among the number of deaths caused by all types of cancer, liver cancer is fourth after lung cancer, stomach cancer and colon cancer. Liver cancer can be classified into primary liver cancer which occurs first in the liver, and metastatic liver cancer wherein a cancer species that originated in an organ other than the liver has metastasized into the liver. The major primary liver cancers that occur in the liver are hepatocellular carcinoma (HCC) which originates in hepatic cells, intrahepatic bile duct carcinoma (or intrahepatic cholangiocarcinoma) which originates in the epithelial cells of the bile ducts, and cases that can be considered to be combinations thereof. Hepatocellular carcinoma which originates in hepatic cells occurs in 90% or more of the cases of primary liver cancer, and hepatocellular carcinoma is often caused by viral hepatitis (HCV, HBV). The rate of contraction of HCV has always been high in East Asia, including Japan, and this is thought to be the reason for the higher rate of occurrence of hepatocellular carcinoma than in Europe and the United States.

Hepatocellular carcinoma exhibits resistance to chemotherapy and radiotherapy, and surgery is considered to be the only therapy enabling complete remission. In order to provide an effective treatment, it is more important than anything else to discover the cancer early and to treat it while it can still be cured.

In order to enable early detection of hepatocellular cancer, there has been progress towards the development of a detection means using tumor markers. Many cancer detection markers have now been developed for hepatocellular cancer, among which α1-fetoprotein (AFP) and PIVKA-II (protein induced by vitamin K absence or antagonist-II) have been clinically used as tumor markers for hepatocellular carcinoma. Other known tumor markers for liver cancer include, for example, CEA, CA19-9, KMO-1, DuPAN-2, SPan-1, CA50, SLX, basic fetoprotein (BFP), NCC-ST-439, alkali phosphatase isozyme, γ-GTP isozyme, TAP, TPA, β2-microglobulin, ferritin, POA and trypsin inhibitor (Patent Document 1).

For example, in a clinical setting, the AFP and PIVKA-II in serum are measured, and the amounts of expression thereof are used to determine the possibility of development of hepatocellular carcinoma. 26% of hepatocellular carcinoma patients test positive for only PIVKA-II, which is greater than the 9% testing positive for only AFP, and at least 61% of patients test positive for at least one, but 39% of patients still test negative for both. Therefore, the tumor markers that are currently being used cannot be considered to be adequate for diagnosing hepatocellular carcinoma, and the development of a new tumor marker is considered to be needed.

For this reason, examinations for early discovery of hepatocellular cancer currently do not depend on hepatocellular carcinoma markers alone, but also include image testing such as ultrasonic testing, computer tomography (CT) and nuclear magnetic resonance imaging (MRI).

In recent years, many tumor markers for hepatocellular carcinoma consisting of genes or polypeptides that are expressed in hepatocellular carcinoma have been developed. For example, hepatocellular carcinoma tumor markers consisting of genes or polypeptides such as Gla-incomplete blood coagulation factor VII (Patent Document 2), the aldolase β gene, the carbamoyl phosphate synthase I gene, the plasminogen gene, EST51549, the albumin gene, the cytochrome P450 subfamily 2E1 gene, the retinol-binding protein gene and the organic anion transporter C gene (Patent Document 3), the human gene ZNFN3A1 having a zinc finger domain and a SET domain (Patent Document 4), glypican-3 (GPC3) which is a heparan sulfate proteoglycan (Patent Document 5), and development and differentiation enhancing factor 1 (DDEFL1) which is located in chromosomal band 1p36.13 and regulates the re-organization of actin cytoskeletons (Patent Document 6) have been disclosed.

Furthermore, hepatocellular carcinoma tumor markers comprising genes or polypeptides such as the presence or absence of deletions in the chromosomal bands 8p12, 16p13.2-p13.3, 16q23.1-q24.3 or 19p13.2-p13.3 (Patent Document 7), Wnt-1 which encodes a family of secreted cysteine-rich proteins (Patent Document 8), the genes MGC47816 for carbamoyl-phosphate synthase L chain and HES6 for a protein containing a helix-loop-helix domain and orange domain (Patent Document 9), cell-associated hepatocellular carcinoma (HCC) proteins including SEMA5A (semaphorin 5A), SLC2A2 (solute carrier family member), ABCC2 (ATP-binding cassette subfamily C member 2) and HAL (histidine ammonia lyase) (Patent Document 10), and human α2,6-sialic acid transferase (Patent Document 11) have been disclosed.

However, methods that detect the occurrence of liver cancer by means of liver cancer tumor markers comprising genes or polypeptides that are expressed in liver cancer are difficult to apply when using serum or bile as the test sample, and in view of the complicated operations that are required for detecting the expression of genes and the need for sensitivity and precision of cancer detection or differential diagnosis of cancer species, these methods have many constraints as detection means for the early detection and diagnosis of liver cancer that can be accurately and conveniently used at the site of medical treatment, and cannot be considered to be entirely satisfactory.

As mentioned above, many cases of hepatocellular carcinoma are caused by viral hepatitis (HCV, HBV). Particularly in the case of HCV (the hepatitis C virus), contraction is followed by acute viral hepatitis, then chronic viral hepatitis, and after a long period of time (about 20 years), hepatic cirrhosis occurs, and this is frequently followed by cancer. With hepatic cirrhosis, repeated inflammation and regeneration occurs, as a result of which normal liver tissue is reduced and the liver changes to an organ that is constituted from fibrous tissue. In the case of HCV and HBV patients, the rate of development of cancer from chronic hepatitis is about 0.8% to 0.9% yearly for mild chronic hepatitis (F1) or moderate chronic hepatitis (F2), but becomes 3.5% yearly for severe chronic hepatitis (F3), and the probability of developing cancer from hepatic cirrhosis (F4) rises to 7% yearly. Additionally, as the pathological condition of the hepatic disease progresses, the chronic hepatitis begins to erode the functions of the hepatic tissue, fibrosis progresses, and as the hepatic cirrhosis becomes complete, hepatocellular carcinoma occurs. In other words, the liver in which the hepatocellular carcinoma occurs is in a highly progressed state of fibrosis, so markers that are affected by reduced liver function and fibrosis lack cancer specificity, and do not lead to early discovery of liver cancer.

In a series of studies in recent years, it has been reported that, in the serum or in hepatocyte precursor cells in hepatocellular carcinoma patients, the activities of glycosyltransferases that synthesize specific glycan structures are elevated or reduced, and that glycan structures that are not observed in normal mature hepatic cells are expressed.

Regarding the hepatocellular carcinoma marker AFP (α1-fetoprotein), a glycan isomer having an α1→6 fucosylated glycan is known as a glycan marker called AFP-L3 fraction due to its reactivity with LCA lectin. AFP-L3 fraction more strongly reflects the occurrence of cancer so that the numerical values are boosted, and thus it is known that the accuracy (specificity) of diagnosis of hepatocellular carcinoma can be raised by measuring the proportion of the L3 fraction in the AFP in blood. However, in a high proportion of hepatocellular carcinoma patients, the AFP level does not increase, in which case the L3 fraction also does not increase, so it cannot be considered to be sufficiently effective as a hepatocellular carcinoma marker, and it has yet to fully satisfy medical needs. On the other hand, it is known that fucosylation is enhanced by the occurrence of hepatocellular carcinoma in liver state changes associated with hepatic fibrosis. For example, there have been numerous reports of fucosylation in AGP (α1-acidic glycoprotein), which is known as a hepatic fibrosis marker. However, enhanced fucosylation of AGP has been widely observed in cancer patients in general, so its specificity to hepatocellular carcinoma is low, and it is difficult to set a cut-off value.

A hepatocellular carcinoma marker that focuses on constituent glycan groups in the glycoproteins in serum has also been disclosed (Patent Document 12). The document indicates that trisialyl glycans that are eliminated or reduced with the onset of hepatocellular carcinoma are labeled and used as hepatocellular carcinoma markers for detecting hepatocellular carcinoma, and that the amount of the hepatocellular carcinoma marker prepared from a sample is fractionated with an anionic exchange column, and calculated by analysis with an elution pattern by high-speed liquid chromatography using an ODS silica column.

Recently, in the search for cancer markers including hepatocellular carcinoma markers, strategies that make use of multiple advanced technologies such as glycoproteomics, lectin microarrays or antibody-overlay lectin microarrays to perform comprehensive search and validation of marker candidate molecules have been proposed (Non-patent Document 1, Patent Document 13). It has been shown that the amounts of indicator glycan markers can be measured in serum being tested, and that hepatic cirrhosis and hepatocellular carcinoma can be discriminated by using a calibration curve. There are several examples of such results.

However, almost all hepatocellular carcinoma marker development is limited to fucose-containing glycoproteins, and these are all basically the same as conventional hepatocellular carcinoma markers in that they discriminate based on differences in the amounts of the markers present in the serum, on the assumption that the expression of glycans will increase with the degree of fibrosis progression in hepatic tissue. Even if they are excellent serum-based indicators of the pathological condition or degree of fibrosis progression in hepatic disease, they cannot be considered to be indicators that are capable of precisely differentially diagnosing hepatocellular carcinoma from hepatic cirrhosis, with performance superior to that of AFP-L3.

When considering the glycan moieties of these conventional hepatocellular carcinoma markers composed of glycans or glycoproteins, they are almost all fucoses, particularly “fucose α1→6 glycans” and “fucose α1→3 glycans”, and these “fucose-containing glycoproteins” are mainly used as the indicators of hepatocellular carcinoma (many documents such as Non-patent Documents 4-8). As mentioned above, among the hepatocellular carcinoma markers that are currently in clinical use, glycan isomers of α-fetoprotein (AFP) that have an α1→6 fucosylated glycan (known as AFP-L3 fraction) detect hepatocellular carcinoma with the highest precision.

An extensive comparative glycan analysis between the proteins in the serum of healthy individuals and hepatocellular carcinoma cell lines that was previously carried out by the present inventors revealed that fucose is a glycan modifier that characterizes hepatic disease, and a group of multiple fucose-containing glycoproteins was identified as markers indicating hepatic disease pathology (Non-patent Document 6, Non-patent Document 14).

These hepatic disease pathology-indicating markers are a group of proteins that can all identify the fibrosis of hepatic tissue that progresses with the disease state from a healthy state of the liver to viral infection, chronic hepatitis and hepatic cirrhosis, and the majority thereof can be used as excellent markers for investigating the fibrosis of the liver and hepatic cirrhosis (Non-patent Documents 7 and 8). However, they are not suitable for use as hepatocellular carcinoma markers capable of clearly distinguishing between hepatocellular carcinoma and hepatic cirrhosis (Non-patent Document 8).

This agrees with reports of the enhanced expression of α1→6 fucosylation enzyme (FUT8), which is an enzyme that causes α1→6 fucosylation, in hepatic tissues in both chronic hepatitis and hepatic cirrhosis (Non-patent Document 2). Though it has been confirmed that the amount of GDP-fucose, which is an enzyme donor substrate, is increased in cancerous portions in human hepatic cancer tissue (Non-patent Document 3), the rate of increase is only about double, and it is not suitable for use as a marker in blood.

On the other hand, while it is a fact that the actual blood concentration of AFP-L3 is significantly elevated in cancer, this could not be explained by the above-mentioned synthesis mechanism alone. Subsequent research has led to the theory that the blood concentration of not only AFP-L3 fraction, but also a group of many specific fucose-containing glycoproteins, is not raised by expression in the hepatocellular carcinoma itself, but rather that the blood concentration is raised (the secretion pathway is changed) due to the cancer spreading in the hepatic cells and causing fucosylated proteins in the hepatic cells, which had been (polar) transported and secreted into the bile ducts (bile), to instead be transported and released into the blood vessels (into the blood), thereby raising the blood concentration (Non-patent Documents 4 and 5). In that case, even if an increase in the expression of fucosylated proteins is observed in the blood, this does not directly reflect the level of advancement of cancer in the cells, so fucosylated proteins cannot be used as a therapeutic target.

As described above, in the wake of AFP-L3 fraction, there was a time of extremely active searching and research and development of hepatocellular carcinoma markers targeting “fucose-containing glycoproteins”, particularly “fucose α1→6 glycans” and “fucose α1-3 glycans”. As a result, many hepatocellular carcinoma markers were identified and some success was achieved in fields such as pathological analysis of hepatic diseases, but the work did not result in the discovery of any markers that are superior to AFP (particularly AFP-L3 fraction), which is currently in clinical use due to its accuracy and convenience as a hepatocellular carcinoma marker, and the search has ground to a halt.

Due to this background, there has been a strong demand for a method that is capable of accurately and reliably distinguishing between hepatic cirrhosis and hepatocellular carcinoma and specifically detecting hepatocellular carcinoma.

### RELATED ART DOCUMENTS

### Patent Documents

Patent Document 1: JP 2002-323499 A
Patent Document 2: JP H8-184594 A
Patent Document 3: JP 2004-105013 A
Patent Document 4: JP 2005-511023 A
Patent Document 5: JP 2005-526979 A
Patent Document 6: JP 2005-503176 A
Patent Document 7: JP 2006-94726 A
Patent Document 8: JP 2007-139742 A
Patent Document 9: JP 2007-506425 A
Patent Document 10: JP 2007-534772 A
Patent Document 11: JP 2007-322373 A
Patent Document 12: JP 2007-278803 A
Patent Document 13: WO 2011/007797
Patent Document 14: WO 2011/007764
Patent Document 15: WO 2010/055950
Patent Document 16: WO 2010/010674

### Non-Patent Documents

Non-patent Document 1: Narimatsu, H. et al., FEBS J., 2010 January, 277(1):95-105.
Non-patent Document 2: Noda, K. et al., Hepatology, 1998 October, 28(4):944-52.
Non-patent Document 3: Noda, K., et al., Cancer Res., 2003 Oct. 1, 63(19):6282-89.
Non-patent Document 4: Nakagawa, T., et al., J. Biol. Chem., 2006 Oct. 6, 281(40):29797-806.
Non-patent Document 5: Nakagawa, T., et al., J. Proteome Res., 2012 May 4, 11(5):2798-806.
Non-patent Document 6: Kaji, H., et al., J. Proteome Res., 2013 Jun. 7, 12(6):2630-40.
Non-patent Document 7: Kuno et al., Clin. Chem., 2011 January, 57(1):48-56.
Non-patent Document 8: Ocho et al., J. Proteome Res., 2014 Mar. 7, 13(3):1428-37.
Non-patent Document 9: Hirabayashi, J., et al., Chem. Soc. Rev., 2013 May 21, 42(10):4443-58.
Non-patent Document 10: Matsuda, A., et al., Biochem. Biophys. Res. Commun., 2008 May 30, 370(2):259-63.
Non-patent Document 11: Matsuda, A., et al., Hepatology, 2010 July, 52(1):174-82.
Non-patent Document 12: Quail, D. F., et al., Nat. Med., 2013 November, 19(11):1423-1437.
Non-patent Document 13: Hirabayashi, J., et al., Electrophoresis, 2011 May, 32(10):1118-28.

### SUMMARY OF THE INVENTION

### Problems to be Solved by the Invention

In order to develop an intrinsic hepatocellular carcinoma marker, it is necessary to identify a marker that is due not to structural changes associated with fibrosis, but that is specifically present only in hepatic cancer cells or in the vicinity thereof, and this requires making it possible to visualize that the marker molecule is actually expressed in the cancerous portions (in the cancer cells themselves or in cells that are specifically present in the interstitial regions in the vicinity thereof).

At the present time, there are no examples wherein glycan changes in glycoproteins have been observed to occur specifically in hepatic cancer cells or in cells that are confined to the interstitial regions in the vicinity thereof.

The present invention provides a hepatocellular carcinoma marker that is a marker for detecting hepatocellular carcinoma, that does not depend on changes in the state of the liver, and that first becomes present in the liver due to the occurrence of cancer. More specifically, the present invention provides a glycoprotein as an intrinsic hepatocellular carcinoma marker, based on the discovery of a lectin that can specifically recognize just glycoproteins having glycans that have been confirmed to be clearly present in only cancerous portions, by comparison of hepatocellular carcinoma portions with surrounding non-cancerous portions.

### Means for Solving the Problems

Based on the above, the present inventors came to the conclusion that, in order to develop a marker that detects liver cancer, it is necessary to search for and discover a marker that is not affected by fibrosis or reduced function of the liver, that is more highly specific to cancer and that appears in cancer tissues. More specifically, the present inventors came to the conclusion that, after comparing hepatocellular carcinoma which is a primary hepatic cancer with surrounding non-cancerous portions, a glycoprotein having glycans that are confirmed to be clearly present in only cancerous portions would be an intrinsic hepatic cancer marker.

In order to identify glycoproteins that are specifically expressed on the cancer cell surface in hepatocellular carcinoma, there have been previous attempts to find genes that are specifically expressed in cancer cells but are not expressed in normal cells and to find glycoproteins that are specifically expressed in the membrane fractions on cancer cell surfaces, but satisfactory results were not obtained. This time, the present inventors thought of targeting not only glycoproteins that are expressed in hepatocellular carcinoma cells themselves, but also glycoproteins that are secreted by cancer cells and cells constituting cancer tissue, and that are confined to the cancer tissue, including the tumor microenvironment (TME) which includes various cells constituting the cancer tissue, and thus directed their study to glycoprotein analysis in cancer tissue. Using a lectin array that was developed by the present inventors for this purpose, experiments were performed by distinctly fractionating cancer tissue and non-cancer tissue, which have different levels of differentiation, in tissue specimens from hepatocellular carcinoma patients, by means of laser microdissection (LMD), and performing comparative glycan analysis after extracting the glycoproteins present therein.

The lectin array technique is the most highly sensitive glycan analysis technology in the world (Non-patent Document 9), and this analysis method performs well in analyzing glycans in glycoproteins that are present in small numbers of cells or tissues. At present, when handling cultured cells, a method of performing analysis by fractionating components on the surfaces or the interiors of cells has been established, but a definitive fractionation method for extracting proteins from minute quantities of tissue fragments obtained by LMD or the like has yet to be established. In other words, tissue-extracted protein solutions that are to be analyzed contain not only proteins that are present on the surfaces of tissue cells, but also include proteins from outside the cells. There are absolutely no examples of experiments that aim to analyze cancer cells including the interstitial regions in the periphery of the cells. Therefore, even if an analysis using conventional cancer tissue fragments reveals that a certain glycan has different lectin signals in cancer tissue and normal tissue, the glycan is not necessarily limited to being present in the cancer cell or tissue surface, or the interstitial regions in the vicinity of the cancer cells. A means for verifying this is needed, and the present inventors have decided to use a validation method based on lectin stains left on the cancer cell or tissue surfaces by labeled lectins, as previously reported by Matsuda et al. (Non-patent Documents 10 and 11). Furthermore, by using this staining method, it is possible to visualize all glycans on glycoproteins present in hepatocellular carcinoma and peripheral regions thereof, including the interstitial regions in the vicinity of hepatocellular cancer cells (TME) that are being targeted by the present inventors.

By lectin array, the present inventors were able to discover multiple lectins in which high levels of fluorescence were observed specifically in cancerous regions without being present in non-cancerous regions, and therefore attempted to validate the results by labeling the respective lectins and performing lectin staining on the hepatocellular cancer tissues in accordance with the method of Matsuda et al. (Non-patent Documents 8 and 9). As the label in this case, DAB staining was performed using horseradish peroxidase (HRP) which is often used for pathological analysis, but images indicating a clear difference in stain intensity were not able to be obtained in the manner of the numerically significant difference that was obtained by lectin array, and in fact, the staining actually appeared to be stronger in the non-cancerous portions than in the cancerous portions, which was the opposite result from that of the lectin array. Next, the use of fluorescent staining, for which suitable conditions are very difficult to arrange, was attempted as a label, but the initial results did not yield significantly different staining between cancerous portions and non-cancerous portions. Nevertheless, the inventors persisted, and upon further studying the staining conditions, found that, among multiple lectins, only NPA lectin is stained in both cancerous portions and non-cancerous portions, but exhibits significantly different staining patterns (such as distribution and staining intensity). By combining the results for lectin array and the results from lectin staining, it was proven for the first time that NPA lectin is a lectin that reacts with glycans that are specifically present in the cancer cell membranes of primary hepatocellular cancer and a portion of the interstitium in the vicinity thereof.

Observing images of the results from lectin staining, it appears that NPA lectin is reacting with the interiors of the hepatic cells in the case of non-cancerous portions, and in the case of cancerous portions, with not only the cancer cell surfaces, but also specific (immune) cells that are present in the interstitial portions in the periphery of cancer cells (the TME) in cancer tissue. In other words, it is possible that the glycoproteins with which NPA lectin reacts are present inside the cells in the case of normal hepatic cells, but with the onset of hepatocellular carcinoma, are expressed or secreted on the cancer cell surfaces and/or the cell surfaces in the TME in the periphery of cancer cells, and also that the glycan structures of glycoproteins that were originally present on the cell surface are changed to glycans that react with NPA lectin with the onset of hepatocellular carcinoma. Additionally, there is the possibility that, aside from the glycoproteins that are secreted on the hepatocellular carcinoma surfaces, the same or different glycoproteins are secreted on the cell surfaces from immune cells that are specifically present in the TME, or that just the glycan structures are changed. In any case, it can be seen that, with the onset of primary hepatocellular carcinoma, glycoproteins having glycans that react with NPA lectin become present on the cancer cell surfaces and/or in the TME around the cancer cells.

It should be noted that the TME, which is the microenvironment in the vicinity of cancer cells, has recently been found to play an important role in the maintenance, infiltration and metastasis of cancer cells (Non-patent Document 12), and has become the subject of careful scrutiny as a target for cancer treatment, particularly in the case of cancers having an extensive peripheral interstitium as in pancreatic cancer and lung cancer (Patent Document 15).

The glycoproteins that react with NPA lectin in the present invention are likely to be glycoproteins of (immune) cells that are specifically present on the cell membrane surfaces and the TME of hepatocellular carcinoma, and can naturally be used as diagnostic markers for hepatocellular cancer, as well as holding promise as therapeutic targets for hepatocellular carcinoma in the future.

As described above, in the present invention, as a step in a validation study, instead of simply focusing on the presence or absence of staining by lectin staining at cancerous portions and non-cancerous portions, improvements were made to take into consideration the staining intensity and patterns, and as a result, it was discovered for the first time that glycoproteins that exhibit the property of binding to NPA lectin are a group of molecules that satisfy the objective.

NPA lectin is generally known to be a lectin that has reactivity with α-mannosyl residues which are the core structures in N-linked glycans, and that also has high reactivity to “fucose α1→6 glycans” (Patent Document 16, etc.). However, in comparative glycan analysis of glycoproteins present in the tissue regions of cancerous portions and non-cancerous portions by lectin array, LCA lectin (lentil lectin), which is a lectin that similarly recognizes “fucose α1→6 glycans”, yielded the opposite result that the signal in the cancerous portions was lower than that in the non-cancerous portions. In tissue staining under the same conditions as with NPA lectin as well, LCA lectin exhibited entirely different stain images, so the glycan that is recognized by NPA lectin as a glycoprotein specific to hepatocellular carcinoma is not “fucose α1→6 glycan”. Additionally, when considering that the signal was significantly lower at cancerous portions than at non-cancerous portions in the case of ConA (Concanavalin A), which does not react with “fucose α1→6 glycan”, and even among the high-mannose type lectins, has high reactivity with long-chain, mannose-rich glycans, the following statements can be made.

(1) According to many documents such as Non-patent Document 13, NPA is classified as a highly mannose-binding lectin, but intensive specificity analysis (see LfDB “http://jcggdb.jp/rcmg/glycodb/LectinSearch”) shows that the lectin does not have especially strong affinity to so-called high-mannose type glycans having more than five mannoses, that the glycans with which it has high affinity primarily have three mannoses, and that it has particularly high affinity to glycans having at least one GlcNAc and/or Gal bound to mannotriose. Additionally, as also mentioned in Patent Document 16, it binds strongly to complex glycans containing core fucose (fucose α1→6 glycan), and is sometimes classified as a core fucose-recognizing lectin.
(2) LCA basically binds strongly to core fucose-containing glycans, but also binds weakly to high-mannose type glycans as well. In that case, it strongly binds to glycans having more than five mannoses.
(3) ConA is a typical lectin that strongly binds to high-mannose type glycans. It has the characteristic that the binding affinity largely changes depending on the number of mannoses, and prominent binding is exhibited when there are more than seven mannoses.

In view of the above, the characteristics of ligand glycans that were discovered as binding with NPA lectin at this time are inferred to be complex type glycans not containing core fucose (fucose α1→6 glycan) and having three (no more than four) mannoses.

In other words, the glycoproteins that were discovered as being primary hepatocellular carcinoma markers in the present invention can be considered to be “NPA lectin-binding glycoproteins” that are further “NPA lectin-binding glycoproteins that do not contain core fucose”.

Alternatively, since the properties of binding to NPA lectin and to LCA lectin and ConA are independent factors, the glycoproteins could also be expressed as being “NPA lectin-binding glycoproteins that do not depend on the property of binding to LCA lectin” or “NPA lectin-binding glycoproteins that do not depend on the property of binding to LCA lectin and ConA”.

The above results indicate that “core fucose”, which has been verified to increase in serum in conjunction with fibrosis of hepatic tissue, is not directly associated with the occurrence of primary hepatocellular carcinoma. In other words, the results also suggest that the transformation from hepatic cirrhosis to hepatocellular carcinoma is not continuous, but occurs through some sort of sudden change.

Additionally, since the glycoproteins that are recognized by NPA lectin and that serve as hepatocellular carcinoma markers are clearly present, not inside the cancer cells, but on the cancer cell surfaces and the peripheral interstitial areas (TME), they are very likely to be secreted glycoproteins. This means that the glycoproteins are likely to also be secreted in blood or in other body fluids, and unlike glycoproteins that are present in blood at high concentrations, such as IgG, are likely to not depend on the property of binding to fucose-recognizing lectins such as LCA lectin, so they can be expected to be capable of differentiating hepatic cirrhosis irrespective of the fibrosis state, even by a less invasive diagnosis of body fluids.

In other words, while conventional hepatocellular carcinoma tests focused on “fucose-containing glycans”, and therefore yielded positives when the reactivity with LCA lectin was low and negatives when the reactivity was high, the present results demonstrate that it is actually likely that primary hepatocellular carcinoma is present in the case of low reactivity with LCA lectin, and they suggest the importance of confirming the reactivity with NPA lectin during primary hepatocellular carcinoma testing.

Furthermore, the primary hepatocellular carcinoma markers comprising “an NPA lectin-binding glycoprotein that does not depend on the property of binding to LCA lectin” can be considered to be glycoproteins that are confined to the cancer cell surfaces or the peripheral TME, and that therefore can serve as therapeutic targets for cancer therapy.

The present invention was completed as a result of making the above-described discoveries.

That is to say, the present invention includes the following.

[1] A hepatocellular carcinoma marker comprising an NPA lectin-binding glycoprotein having an NPA lectin-binding glycan epitope that has at least one of the following properties (1) to (5):
(1) the glycan epitope does not include core fucose (fucose α1→6 glycan);
(2) the glycan epitope comprises a complex-type glycan having three (four or fewer) mannoses;
(3) the glycan epitope does not include a high-mannose-type glycan having five or more mannoses;
(4) the glycan epitope comprises a complex-type glycan that does not depend on the property of binding to LCA lectin; and
(5) the glycan epitope comprises a complex-type glycan that does not depend on the property of binding to ConA lectin.
[2] The hepatocellular carcinoma marker according to [1], wherein the glycoprotein is a glycoprotein that is present on the surfaces of cancer cells in hepatic tissue, or is present in the interstitium in the vicinity of the cells.
[2′] The hepatocellular carcinoma marker according to [1] or [2], for use in a method for detecting hepatocellular carcinoma, wherein the method comprises a step of obtaining a biological sample from a subject.
[3] The hepatocellular carcinoma marker according to [1] or [2], wherein the glycoprotein is a glycoprotein chosen from among complement factor H (CFH), fibrillin-1 (FBN1), fibronectin (FN1), oxygen-regulated protein (HYOU1), epidermal growth factor receptor (EGFR), prosaposin (PSAP), cathepsin D (CTSD) and lysosome-associated membrane protein 2 (LAMP-2).
[4] A detection reagent for detecting the hepatocellular carcinoma marker according to any one of [1] to [3], wherein the detection reagent contains NPA lectin.
[5] The detection reagent according to [4], further comprising LCA lectin or ConA lectin.
[6] A detection reagent for detecting the hepatocellular carcinoma marker according to any one of [1] to [3], wherein the detection reagent contains an antibody that binds to at least one NPA lectin-binding glycoprotein chosen from among complement factor H (CFH), fibrillin-1 (FBN1), fibronectin (FN1), oxygen-regulated protein (HYOU1), epidermal growth factor receptor (EGFR), prosaposin (PSAP), cathepsin D (CTSD) and lysosome-associated membrane protein 2 (LAMP-2).
[7] A method for detecting hepatocellular carcinoma, wherein hepatocellular carcinoma is detected by in vitro detection of the hepatocellular carcinoma marker according to any one of [1] to [3] in a test sample.
[8] The method according to [7], wherein the in vitro detection of the hepatocellular carcinoma marker is performed by NPA staining of test cells or tissues using a labeled NPA lectin.
[9] The method according to [7], wherein the in vitro detection of the hepatocellular carcinoma marker is performed by using a lectin array analysis method using a lectin array including NPA lectin, or by a lectin-antibody ELISA method including NPA lectin.
[10] The method according to [9], wherein the lectin array analysis method uses a lectin array containing at least LCA lectin or ConA lectin in addition to NPA lectin.
[11] The method according to [9], wherein the lectin-antibody ELISA method is a method for detecting the hepatocellular carcinoma marker by a sandwich method using NPA lectin and an antibody that binds to an NPA lectin-binding glycoprotein, the method being performed by immobilizing the antibody that binds to an NPA lectin-binding glycoprotein on a support, and using a lectin overlay wherein the NPA lectin-binding glycoprotein which is the hepatocellular carcinoma marker is sandwiched by a labeled NPA lectin, or using an antibody overlay wherein the NPA lectin-binding glycoprotein which is the hepatocellular carcinoma marker is sandwiched by a labeled antibody.
[12] The method according to [11], wherein the antibody that binds to the NPA lectin-binding glycoprotein is an antibody that binds to at least one glycoprotein chosen from among CFH, FBN1, FN1, HYOU1, EGFR, PSAP, CTSD and LAMP-2.
[13] The method according to any one of [7] and [9] to [12], wherein the in vitro detection of the hepatocellular carcinoma marker is performed by using a blood sample containing serum components as the test sample, the method comprising a preliminary step of adsorbing the test sample to an α-2,6-sialic acid-binding lectin immobilized on a support, and a step of obtaining a fraction that is not adsorbed to the α-2,6-sialic acid-binding lectin.
[14] The method according to [13], wherein the α-2,6-sialic acid-binding lectin is at least one lectin chosen from among SNA, SSA, TJAI and PSL1a lectin.
[15] A measurement method for determining the presence of hepatocellular carcinoma or a level of progression or malignancy of carcinoma, the measurement method comprising:

a step of measuring, in a test sample obtained from a hepatic tissue being tested, the reactivity of the test sample to lectins including NPA lectin, by using a lectin-antibody ELISA method or a lectin array analysis method including NPA lectin.

[16] The measurement method according to [15], wherein the measurement method comprises:
(1) a step of preparing a discrimination formula or a calibration line corresponding to the level of progression or malignancy of hepatocellular carcinoma, by taking preliminary measurements of the reactivity of a plurality of hepatocellular carcinoma tissues and normal tissues to lectins including NPA lectin, using the lectin array analysis method or the lectin-antibody ELISA method; and
(2) a step of determining the presence of hepatocellular carcinoma or the level of progression or malignancy of carcinoma by fitting, to the discrimination formula or the calibration line, measurement values of the reactivity of the test sample to lectins including NPA lectin.
[17] A measurement method for determining the presence of hepatocellular carcinoma or a level of progression or malignancy of carcinoma, using a serum-containing sample as a test sample, the measurement method comprising the following steps to be performed on the serum-containing test sample:
(1) a step of causing adsorption to an α-2,6-sialic acid-binding lectin immobilized on a support;
(2) a step of obtaining a fraction that is not adsorbed to the α-2,6-sialic acid-binding lectin; and
(3) a step of measuring the reactivity of the test sample to lectins including NPA lectin, using a lectin-antibody ELISA method or a lectin array analysis method including NPA lectin.
[18] A measurement method for determining the presence of hepatocellular carcinoma or a level of progression or malignancy of carcinoma, the measurement method comprising:

a step of measuring, in a test sample obtained from a hepatic tissue being tested, the reactivity of the test sample to lectins including NPA lectin, by using a sandwich ELISA method involving lectins including NPA lectin and an antibody that binds to at least one glycoprotein chosen from among CFH, FBN1, FN1, HYOU1, EGFR, PSAP, CTSD and LAMP-2.

[19] A method for determining the presence of hepatocellular carcinoma or a level of progression or malignancy of carcinoma using a lectin-antibody ELISA method or a lectin array analysis method including NPA lectin, the method comprising:
(1) a step of preparing a discrimination formula or a calibration line corresponding to the level of progression or malignancy of hepatocellular carcinoma, by taking preliminary measurements of the reactivity of a plurality of hepatocellular carcinoma tissues and normal tissues to lectins including NPA lectin, using the lectin array analysis method or the lectin-antibody ELISA method;
(2) a step of measuring the reactivity of a test sample obtained from a hepatic tissue being tested to lectins including NPA lectin, by subjecting the test sample to the lectin array or ELISA; and
(3) a step of determining the presence of hepatocellular carcinoma or the level of progression or malignancy of carcinoma by fitting measurement values of the reactivity of the test sample to lectins including NPA lectin, obtained in step (2), to the discrimination formula or the calibration line obtained in step (1).
[20] The method according to [19], wherein the lectin array analysis method or the lectin-antibody ELISA method includes NPA lectin and LCA lectin and/or ConA lectin, and the prepared discrimination formula or calibration line further includes a discrimination formula or calibration line for LCA lectin and/or ConA lectin.
[21] A method for determining the presence of hepatocellular carcinoma or a level of progression or malignancy of carcinoma by tissue staining, the method comprising the following steps (1) to (4):
(1) a step of preparing a tissue section of a test sample from a hepatic tissue being tested;
(2) a step of tissue staining using fluorescent-labeled NPA lectin;
(3) a step of observing the presence or absence and the intensity of fluorescence at the cell surfaces and/or the interstitium in the vicinity thereof; and
(4) a step of determining the presence of hepatocellular carcinoma when at least a standard level of fluorescence is observed in step (3) and determining a level of progression or malignancy of the carcinoma in accordance with the intensity thereof.
[21] A kit for tissue staining in order to determine the presence of hepatocellular carcinoma or a level of progression or malignancy of carcinoma, the kit comprising a fluorescent-labeled NPA lectin.
[22] A kit for detecting a hepatocellular carcinoma marker, wherein one of the following (1) and (2) is immobilized on a support, and the other is labeled:
(1) a lectin including NPA lectin; and
(2) an antibody that binds to at least one glycoprotein chosen from among CFH, FBN1, FN1, HYOU1, EGFR, PSAP, CTSD and LAMP-2.
[22′] A kit for detecting a hepatocellular carcinoma marker in order to determine the presence of hepatocellular carcinoma or a level of progression or malignancy of carcinoma, wherein one of the following (1) and (2) is immobilized on a support, and the other is labeled:
(1) a lectin including NPA lectin; and
(2) an antibody that binds to at least one glycoprotein chosen from among CFH, FBN1, FN1, HYOU1, EGFR, PSAP, CTSD and LAMP-2.
[23] A kit for detecting a hepatocellular carcinoma marker, wherein the kit uses at least NPA lectin, and further uses LCA lectin and/or ConA lectin.
[23′] A kit for detecting a hepatocellular carcinoma marker in order to determine the presence of hepatocellular carcinoma or a level of progression or malignancy of carcinoma, wherein the kit uses at least NPA lectin, and further uses LCA lectin and/or ConA lectin.
[24] The kit according to [22] or [23], wherein the kit uses a serum-containing sample as a test sample, and further comprises an α-2,6-sialic acid-binding lectin.
[25] Use of the hepatocellular carcinoma marker according to any one of [1] to [3] in the production of a kit for detecting a hepatocellular carcinoma marker.
[26] Use of the hepatocellular carcinoma marker according to any one of [1] to [3] in the production of a kit for determining the presence of hepatocellular carcinoma or a level of progression or malignancy of carcinoma.

### Effects of the Invention

The present invention provides an intrinsic hepatocellular carcinoma marker comprising “an NPA lectin-binding glycoprotein that does not depend on the property of binding to LCA lectin”, that first becomes present with the occurrence of hepatocellular carcinoma, without depending on fibrosis or reduced function in the liver, and also provides a method for detecting the hepatocellular carcinoma marker by means of a kit including NPA lectin. Additionally, the present invention makes it possible to differentiate hepatocellular carcinoma from hepatic cirrhosis that is unrelated to the progression of hepatic fibrosis or reduced function, by the detection of the hepatocellular carcinoma marker, and further opens the road to the development of drugs or the development of therapies for the treatment of hepatocellular carcinoma, by using the hepatocellular carcinoma marker, which is localized on cancer cell surfaces and the surrounding TME, as a target.

### BRIEF DESCRIPTION OF THE DRAWINGS

FIG. 1 Serially sectioned samples (hematoxylin-eosin stained: top) surgically resected from a hepatocellular carcinoma patient and samples after LMD (hematoxylin stained: bottom).

FIG. 2 Comparative glycan analysis results using lectin array of hepatic tissue samples from HCV-infected hepatocellular carcinoma patients.

FIG. 3 Comparative glycan analysis results of hepatic tissue samples from non-HCV- and non-HBV-infected hepatocellular carcinoma patients.

FIG. 4 Top ten N-type glycans binding to each lectin.

FIG. 5 Performance comparison between lectin array and sandwich ELISA using model cell lines.

FIG. 6 Performance comparison between lectin array and sandwich ELISA using protein solutions from tissues of hepatocellular carcinoma patients.

FIG. 7 Hematoxylin-eosin stains (left) and NPA lectin stains (right) of hepatic tissue samples surgically resected from a hepatocellular carcinoma patient.

FIG. 8 Narrow-view image (×60 oil immersion lens) of lectin stains in hepatic tissue samples, showing non-cancerous portions (top) and moderately differentiated cancerous portions (bottom) from the same tissue.

FIG. 9 Lectin array and sandwich ELISA using tissue lysates from cancerous portions and non-cancerous portions of tissues from hepatocellular cancer patients (7 cases) (wherein solid bars denote cancerous portions and open bars denote non-cancerous portions).

FIG. 10 Lectin signal comparisons of culture supernatants in AFP-producing cell lines and AFP-non-producing cell lines among hepatocellular carcinoma culture cell lines.

FIG. 11 α-2,6-sialic acid-recognizing lectin reactivity of NPA-binding glycoproteins in culture supernatant of AFP-producing cell lines and AFP-non-producing cell lines among hepatocellular carcinoma culture cell lines.

FIG. 12 Lectin analysis of SSA-non-adsorbing NPA-adsorbing fractions of serum from non-HBV and non-HCV patients to which a multi-step lectin-using method has been applied.

FIG. 13 Western blotting diagrams showing the presence of HYOU1, EGFR, PSAP, CTSD and LAMP-2 glycoproteins in an NPA lectin elution fraction in cell extracts from Huh7, HAK 1A and HLF cell lines.

FIG. 14 Western blotting diagrams showing the presence of CFH, FN1, PSAP, CTSD and LAMP-2 glycoproteins in an NPA lectin elution fraction in culture supernatants of serum-free cultures from Huh7, HAK 1A, HAK 1B, KYN-1 and HLF cell lines.

FIG. 15 Antibody-lectin sandwich ELISA diagram showing the presence of FBN1 and FN1 glycoproteins in NPA lectin elution fractions of culture supernatants of serum-free cultures of HuH-7, HAK 1B and KYN-1 cell lines, and antibody-lectin sandwich ELISA diagram showing the presence of CTSD, PSAP and LAMP-2 glycoproteins in NPA lectin elution fractions of culture supernatants of serum-free cultures of a HAK 1A cell line. Detection was performed by a sandwich ELISA measurement system using biotin-labeled NPA lectin wherein anti-FBN antibody and FN1 antibody were immobilized on a plate.

FIG. 16 Western blotting diagram showing the presence of CTSD glycoproteins in an immunoprecipitation elution fraction using an anti-CD9 antibody or an anti-CD81 antibody for the culture supernatant of a serum-free culture of a HAK 1A cell line.

### MODES FOR CARRYING OUT THE INVENTION

### 1. Regarding the Hepatocellular Carcinoma Marker According to the Present Invention

### (1-1) Glycoproteins Serving as the Hepatocellular Carcinoma Marker of the Present Invention

The hepatocellular carcinoma marker of the present invention can be described as an “NPA lectin-binding glycoprotein” that is further an “NPA lectin-binding glycoprotein that does not contain core fucose (fucose α1→6 glycan)”. More specifically, it can be described as a “glycoprotein that does not contain core fucose (fucose α1→6 glycan) and that has a complex type glycan having three (no more than four) mannoses”. Additionally, it can be described as an “NPA lectin-binding glycoprotein that does not contain, in the epitope, core fucose (fucose α1→6 glycan) or a glycan having five or more mannoses”. Other characteristics of the glycans are described in (1-3) below.

Additionally, the glycoprotein definitely reacts with NPA lectin, but does not depend on the property of binding to LCA lectin, which exhibits similar behavior in connection with the property of binding to core fucose (fucose α1→6 glycan), so the hepatocellular carcinoma marker of the present invention can also be described as an “NPA-binding glycoprotein that does not depend on the property of binding to LCA lectin”. Furthermore, since it also does not depend on the property of binding to ConA lectin, which is often classified among the same high-mannose type lectins, it can also be described as an “NPA lectin-binding glycoprotein that does not depend on the property of binding to ConA and LCA lectin”.

Summarizing the above, the hepatocellular carcinoma marker of the present invention can be accurately described as:

“A hepatocellular carcinoma marker comprising an NPA lectin-binding glycoprotein having an NPA lectin-binding glycan epitope that has at least one of the following properties (1) to (5):

(1) the glycan epitope does not include core fucose (fucose α1→6 glycan);
(2) the glycan epitope comprises a complex-type glycan having three (no more than four) mannitoses;
(3) the glycan epitope does not include a high-mannose-type glycan having five or more mannoses;
(4) the glycan epitope comprises a complex-type glycan that does not depend on the property of binding to LCA lectin; and
(5) the glycan epitope comprises a complex-type glycan that does not depend on the property of binding to ConA lectin.”

As typical descriptions, the hepatocellular carcinoma marker of the present invention will hereinafter be described as a “glycoprotein comprising an NPA lectin-binding glycan epitope that does not include core fucose” or simply an “NPA lectin-binding glycoprotein that does not include a core fucose” or an “NPA lectin-binding glycoprotein”.

Furthermore, the glycoproteins that serve as the hepatocellular carcinoma marker of the present invention, when considering the results of tissue staining, are glycoproteins that are confined to the cell membrane surfaces and immune cells in the environs of the cancer cells (TME) in hepatocellular carcinoma. Furthermore, there is the possibility that the glycoproteins are glycoproteins that are present intracellularly in organelles or the like when the cells are normal, but come to be secreted extracellularly with the development of hepatocellular carcinoma. As other possibilities, they may be secreted extracellularly by being cleaved by a protease, or may be present on the surface or contained inside a secretory vesicle such as an exosome.

In other words, by focusing on the location where it is present, the hepatocellular carcinoma marker of the present can also be described as an “NPA lectin-binding glycoprotein that is specifically present on the cell membrane surfaces of hepatocellular carcinoma and/or in immune cells in the TME”.

### (1-2) Regarding the Lectins Used in the Present Invention

(a) Lectin for directly detecting glycans from the hepatocellular carcinoma marker of the present invention:

The lectin that directly recognizes glycan epitopes in the glycans from glycoproteins (NPA-binding proteins) that serve as the hepatocellular carcinoma marker in the present invention is NPA lectin.

### <NPA Lectin>

NPA lectin refers to a lectin belonging to the monocot mannose-binding lectin family that is found in wild daffodils (Narcissus pseudonarcissus). It is sometimes referred to as NPL lectin. Here, “lectin” is defined as “a protein that specifically recognizes, and binds and cross-links with a glycan”.

While NPA lectin can be extracted from wild daffodils, then isolated and purified, it is already commercially available from EY Laboratories, Inc. Biotinylated NPL is also available from Vector Laboratories, Inc.

NPA lectin has monosaccharide specificity to Man. According to detailed specificity analysis (see the LfDB), for which the top ten glycans are shown in FIG. 4, NPA lectin does not have such strong affinity to so-called high-mannose type glycans having five or more mannoses, but has high affinity primarily to glycans having three mannoses, with particularly high affinity to glycans having at least one GlcNAc and/or Gal bound to mannotriose.

Additionally, it binds strongly to complex type glycans containing core fucose (fucose α1→6 glycan), and is sometimes treated as being in the same group as “core fucose-recognizing lectins” such as LCA lectin (Patent Document 16).

(b) Lectins for confirming that a glycan is not a glycan from a glycoprotein (NPA-binding protein) that serves as the hepatocellular carcinoma marker in the present invention:

The glycan epitopes in the glycans from the glycoproteins serving as the hepatocellular carcinoma marker in the present invention are characterized by not including core fucose (fucose α1→6 glycan) and not including a high-mannose type having five or more mannoses.

Therefore, lectins that have high affinity to fucose α1→6 glycan and do not have affinity to trimannose-containing glycans, or lectins that have high affinity to high-mannose type glycans having five or more mannoses can serve as so-called “negative markers” indicating that a glycoprotein bound by NPA lectin is not a glycoprotein that serves as a hepatocellular carcinoma marker. Typical examples of such lectins include, for the former, “LCA or PSA, AOL or AAL lectin”, particularly “LCA lectin”, and for the latter, “ConA lectin”.

### <LCA Lectin>

LCA lectin is a lectin belonging to the legume lectin family, obtained from lentils (Lens culinaris), and having monosaccharide specificity to Man and Glc. LCA lectin, for which the top ten is shown in FIG. 4, basically binds strongly to glycans containing core fucose. Aside therefrom, it also binds weakly to high-mannose type glycans, and among high-mannose type glycans, binds strongly to those having five or more mannoses.

LCA lectin is commonly used and has become standard as a lectin having high affinity to typical glycoproteins containing core fucose (fucose α1→6 glycan) (Patent Document 16, etc.). Lectin columns having LCA lectin bound thereto are commercially available, and are used as lectin affinity chromatography kits for separating and purifying glycoproteins (Science Tools from Amersham Biotech 3, 3 (1998) p. 5-6).

### <ConA Lectin>

ConA (Concanavalin A) is a lectin belonging to the legume lectin family, obtained from Canavalia ensiformis in the legume family, having monosaccharide specificity to Man and Glc. ConA is representative of lectins that strongly bind to high-mannose type glycans. Lectin columns having ConA bound thereto are commercially available, and are used along with LCA lectin columns as lectin affinity chromatography kits for separating and purifying glycoproteins (Science Tools from Amersham Biotech 3, 3 (1998) p. 5-6).

The affinity of ConA largely varies depending on the number of mannoses, and it has the characteristic of exhibiting prominent binding when there are seven or more mannoses.

(c) Regarding lectins that have the possibility of increasing hepatocellular carcinoma marker detection accuracy by being used in combination with NPA lectin:

### <DSA Lectin>

DSA lectin is a lectin obtained from Datura stramonium, having specific affinity to Galβ1→4GlcNAc, and having significantly (p<0.001) high reactivity at cancerous portions of about the same level as NPA lectin, according to lectin array analysis (FIG. 2) of cancerous portions and non-cancerous portions from hepatic tissue specimens from HCV-infected hepatocellular carcinoma patients. In other words, a glycoprotein comprising a complex type glycan having three or more Galβ1→4GlcNAc on a non-reducing terminal, which is recognized by DSA lectin, can also be considered to be a hepatocellular carcinoma marker candidate. However, in view of the fact that, in the case of DSA lectin, the value was high even at non-cancerous portions, the glycoprotein is not one that is first expressed or biosynthesized with the occurrence of hepatocellular carcinoma, but rather simply increases with the occurrence of cancer, so it cannot be a hepatocellular carcinoma marker candidate in the intrinsic sense. Therefore, the present invention does not directly pertain thereto. However, when used in combination with the NPA lectin of the present invention, there is a possibility that the detection accuracy can be raised.

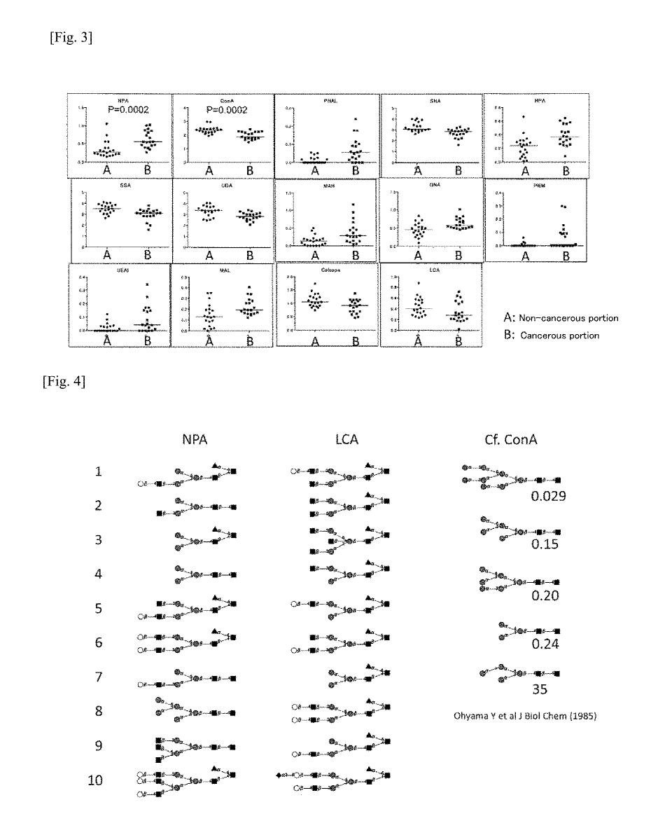

Additionally, FIG. 3 shows lectin array analyses (FIG. 3) of cancerous portions and non-cancerous portions from hepatic tissue specimens from non-HCV- and non-HBV-infected hepatocellular carcinoma patients, wherein HPA lectin, as with NPA lectin, exhibited a high value in cancerous portions and a low value in non-cancerous portions with a significant difference, but the value was still high at the non-cancerous portions, and so the present invention does not directly pertain thereto. However, as in the case of DSA lectin, there is a possibility that the detection accuracy can be raised by use in combination with the NPA lectin of the present invention, and further in combination with DSA lectin.

(d) Lectins for enriching NPA-binding proteins in serum

Additionally, when implementing the hepatocellular carcinoma marker detection of the present invention using a blood sample such as serum, the detection efficiency of the hepatocellular carcinoma marker can be raised by concentrating the NPA-binding proteins, by preliminarily removing glycoproteins having an α-2,6-sialic acid (Neu5Ac α2-6Gal or Neu5Gc α2-6Gal), which are abundant in serum.

α-2,6-sialic acid has been observed to have increased expression at the surfaces of various types of cancer cells of high malignancy, and there have been reports that increased expression of α-2,6-sialic acid in N-binding glycoproteins is associated with progression, metastasis and poor prognoses for cancer (Cancer Res., 2013 Apr. 1; 73(7) 2368-78). However, in the case of hepatocellular carcinoma, increased expression of α-2,6-sialic acid is not observed in a systematic way, and increases or decreases in α-2,6-sialic acid do not necessarily serve as marker indicators for the hepatocellular carcinoma marker glycoprotein (NPA-binding glycoprotein) of the present invention, and NPA itself does not exhibit the property of binding to α-2,6-sialic acid-binding glycans, so it is the glycans not containing α-2,6-sialic acid that can be considered to serve as serum markers.

On the other hand, the glycoproteins in serum include many glycoproteins that originally bind to NPA, even in the case of serum from normal individuals. However, the present invention has revealed that such glycoproteins from normal cells often simultaneously include α-2,6-sialic acid.

For the above reasons, when detecting and measuring the hepatocellular carcinoma marker glycoproteins (NPA-binding glycoproteins) of the present invention using a serum-containing sample, providing a preliminary step of reacting the serum-containing sample with a lectin (SNA, SSA, TJAI or PSL1a lectin) that specifically recognizes α-2,6-sialic acid so as to remove the α-2,6-sialic acid-containing glycoproteins has the effect of markedly reducing the background and is advantageous. For example, the serum test sample can be processed with an affinity column or magnetic bead column to which these α-2,6-sialic acid-recognizing lectins are immobilized. While most of the proteins in the serum are trapped in the column, the hepatocellular carcinoma markers of the present invention (NPA-binding glycoproteins) will pass right through, and will consequently be enriched.

Examples of the lectin to be used in this case include SNA, SSA, TJAI and PSL1a lectin, but as an alternative to these lectins, a known anti-α-2,6-sialic acid antibody (Cancer Res., 2013 Apr. 1; 73(7) 2368-78) can also be used.

### <TJAI Lectin>

TJAI lectin (Trichosanthes japonica lectin-I) can be extracted from Trichosanthes japonica, but is also commercially available from Seikagaku Corp.

### <SSA Lectin>

SSA lectin (Sambucus sieboldiana lectin) can be extracted from Sambucus sieboldiana, but is also commercially available from Seikagaku Corp.

### <SNA Lectin>

SNA lectin (Sambucus nigra lectin) can be extracted from Sambucus nigra, but is also commercially available from Vector Laboratories.

### <PSL1a Lectin>

PSL1a lectin (Polyporus squamosus lectin) can be extracted from Polyporus squamosus, but rPSL1a lectin, which is a recombinant having α-2,6-sialic acid specificity, is commercially available from Wako Pure Chemical Industries.

(e) Regarding other lectin information:

Information regarding lectins is available at the websites of the Lectin Frontier Database (LfDB) and the Biotechnology Research Institute for Drug Discovery of the National Institute of Advanced Industrial Science and Technology of Japan.

### (1-3) Characteristics of the Glycans in the Hepatocellular Carcinoma Markers of the Present Invention

The greatest characteristic of the glycans in the hepatocellular carcinoma markers of the present invention is that the glycans do not depend on binding to LCA lectin, which has extremely high affinity to core fucose (fucose α1→6 glycan), so it is likely that the glycans do not include core fucose (fucose α1→6 glycan). At least, the glycan epitopes in the glycoproteins that serve as the marker in the present invention can be considered as not including core fucose (fucose α1→6 glycan).

Additionally, the glycans in the hepatocellular carcinoma marker of the present invention can be considered to be characterized in that the glycans do not depend on binding to ConA, which has an extremely high affinity to high-mannose type glycans having five or more mannoses, specifically in that the glycans are not high-mannose type glycans having five or more mannoses, or are complex type glycans having three (no more than four) mannoses. Additionally, high-mannose type glycans having five or more glycans can be considered as not serving as epitopes for the marker of the present invention.

In other words, the glycoproteins that serve as the primary hepatocellular carcinoma marker discovered in the present invention can be described as an “NPA lectin-binding glycoprotein” that is further a “glycoprotein comprising an NPA lectin-binding glycan that does not include core fucose”, or a “glycoprotein comprising an NPA lectin-binding glycan that does not include a high-mannose type glycan having five or more mannoses”. They can also be described as a “glycoprotein that does not include core fucose, that comprises a complex type glycan having three (no more than four) mannoses, and that comprises an NPA lectin-binding glycan”. They can also be described as an “NPA lectin-binding glycoprotein comprising a glycan epitope that does not include core fucose or a high-mannose type glycan having five or more mannoses”.

2. Glycoproteins that Serve as the Hepatocellular Carcinoma Marker of the Present Invention and Antibodies Specific Thereto
(2-1) NPA Lectin-Binding Glycoproteins that Serve as the Hepatocellular Carcinoma Marker

The NPA lectin-binding glycoproteins of the present invention can be considered to be glycoproteins that are specifically present in the cancer cells and the interstitial portions in the vicinity thereof (TME) in the cancerous portions of livers suffering from hepatocellular carcinoma, so it is clear that a significant quantity is present in hepatocellular carcinoma tissue resected from hepatocellular carcinoma patients. Therefore, such hepatocellular carcinoma tissue that is to be treated as waste can be collected in large quantities, and protein fractions can be obtained from these cancer tissues using known methods. Since large quantities can be easily obtained by means of lectin chromatography or the like having NPA lectin immobilized thereto, the amino acid sequences and glycan structures of the obtained glycoproteins can be determined as needed.

In the present invention, the Lectin-IGOT-LC/MS method, previously developed by the present inventors (Japanese Patent No. 4220257; Kaji, H., et al., Nature Protocols 1, 3019-3027 (2006)), was used as such a method for allowing multiple candidate glycoproteins to be efficiently identified, and eight different hepatocellular carcinoma markers were identified.

These glycoproteins can provide glycan targets for hepatocellular carcinoma diagnosis using serum test samples or test cell sections, and glycan targets for treating hepatocellular carcinoma.

Specifically, as indicated in Table 1, complement factor H (CFH), fibrillin-1 (FBN1), fibronectin (FN1), oxygen-regulated protein (ORP-150, Hypoxia Up-Regulated 1: HYOU1), epidermal growth factor receptor (EGFR), prosaposin (PSAP), cathepsin D (CTSD) and lysosome-associated membrane protein 2 (LAMP-2) were identified. These marker molecules described in Table 1 are NPA-binding glycoproteins having a plurality of N-linked glycans characterized by specifically binding to NPA lectin, and can serve as hepatocellular carcinoma markers that are capable of detecting or determining hepatocellular carcinoma.

Any of the hepatocellular carcinoma markers shown in Table 1 may be used as long as they are hepatocellular carcinoma marker glycoproteins as indicated in Table 1 having a glycan attached to an asparagine residue at a glycosylation site shown in Table 1, or glycoprotein fragments including at least one asparagine residue at a glycosylation site shown in Table 1 to which a glycan is attached. These hepatocellular carcinoma markers may be used singly, or by combining two or more. For example, two or more different hepatocellular carcinoma marker glycoproteins may be used.

By detecting the presence or absence of these hepatocellular carcinoma markers, it is possible to determine the presence of hepatocellular carcinoma and/or the level of progression or malignance of cancer in a test sample.

TABLE 1

| Gene Symbol | NPA-IGOT | N-glycosylation site |
|---|---|---|
| EGFR | + | 11 |
| FN1 | + | 9 |
| FBN1 | + | 15 |
| HYOU1 | + | 9 |
| CFH | + | 9 |
| CTSD | + | 2 |
| LAMP2 | + | 16 |
| PSAP | + | 5 |

<Epidermal Growth Factor Receptor (EGFR)>

Epidermal growth factor receptor (EGFR, ERBB, ERBB1) is a tyrosinase-type receptor that is expressed on the cell membrane surfaces of various types of cells, such as epidermal cells and mesenchymal cells, and is a glycoprotein that is associated with signaling of epidermal growth factor (EGF), which controls cell proliferation and growth. Its overexpression is observed in renal cancer and various types of malignant tumors, and is also known as a poor prognosis factor for cancer.

### <Fibronectin-1 (FN1)>

Fibronectin (FN, FN1, CIG, FINC, GFND2, LETS, MSF) is present in serum as a soluble dimeric glycoprotein, and is present on cell surfaces or in the extracellular matrix as a dimer or a multimer. It has received attention as a canceration-associated factor.

### <Fibrillin-1 (FBN1)>

Fibrillin (FBN1, FBN, MASS, MFS1, OCTD, SGS, WMS) belongs to the fibrillin family and is a large glycoprotein in the extracellular matrix that serves as a protein constituting the 10-12 nm Ca-binding sites of microfibrils.

### <Oxygen-Regulated Protein (ORP-150, Hypoxia Up-Regulated 1: HYOU1)>

Oxygen-regulated protein (HYOU1, Grp170, HSP12A, ORP150) is a protein that belongs to the heat shock protein 70 family, that is involved in folding and secretion of proteins in the endoplasmic reticulum (ER), and that suppresses apoptosis and has a cytoprotective function against perturbation induced by hypoxia. It has also been observed to be highly expressed in breast cancer.

### <Complement Factor H (CFH)>

Complement factor H (CFH, ARMD4, ARMS1, FHL1, HF, HF1, HF2, HUS) is a glycoprotein that is secreted in the blood as a member of complement activation control (RCA), and is involved in the natural protective mechanism against bacterial infections.

### <Cathepsin D (CTSD)>

Cathepsin D (CTSD, CLN10, CPSD) is a type of lysosomal Asp protease, and is a cause of various diseases such as breast cancer and Alzheimer's disease due to mutation of the gene.

### <Lysosome-Associated Membrane Protein 2 (LAMP-2)>

Lysosome-associated membrane protein 2 (LAMP-2, CD107b) belongs to the cell membrane glycoprotein family, has a role in providing selectins with glycoligands, and is associated with cancer metastasis.

### <Prosaposin (PSAP)>

Prosaposin (PSAP, GLBA, SAP1) is a saposin precursor that is cleaved into saposins A, B, C and D. While saposins A to D are localized in the lysosomal compartment, the precursor has neurotrophic activity as a secretory protein or as an integral membrane protein.

### (2-2) Anti-NPA Lectin-Binding Glycoprotein Antibodies for Detecting the Hepatocellular Carcinoma Marker

Antibodies that are specific to the relevant protein moieties can be prepared on the basis of amino acid sequence information for the glycoproteins. Additionally, the glycan structures of the glycan epitopes recognized by NPA lectin can be accurately determined based on the glycan structures of the relevant glycoproteins, so antibodies that recognize the relevant glycan epitopes can be easily obtained using known antibody preparation methods with the relevant glycan epitopes as the immunogens. Furthermore, it is also possible to obtain other lectins or antibodies that recognize glycan structures other than the relevant glycan epitopes.

Furthermore, a hepatocellular carcinoma-specific antibody that simultaneously recognizes a protein moiety and a glycan including the glycan epitope of a relevant glycoprotein can be prepared by using the CasMab method (Kato, Y., et al., Sci Rep., 2014 Aug. 1; 4: 5924, doi: 10.1038/srep05924), so a therapeutic antibody drug that has hepatocellular carcinoma as the therapeutic target can be provided.

In the hepatocellular carcinoma marker detection method and the hepatocellular carcinoma determination method of the present invention, antibodies that specifically bind to the protein moiety of an NPA lectin-binding glycoprotein are particularly effective, and may be used alone, but are preferably used in conjunction with NPA lectin. These antibodies may be polyclonal antibodies, but are preferably monoclonal antibodies, and may be antibody fragments such as Fab as long as their antigen activity is not compromised. These antibodies and fragments thereof will be referred to collectively as anti-NPA lectin-binding glycoprotein antibodies.

Additionally, “anti-NPA lectin-binding glycoprotein antibodies” include cases of antibodies (hepatocellular carcinoma-specific antibodies) that simultaneously recognize the glycan moiety and the protein moiety. The relevant hepatocellular carcinoma-specific antibodies may be very effectively used to detect hepatocellular carcinoma markers and to diagnose hepatocellular carcinoma when used alone, but their accuracy may be further raised by using them in conjunction with antibodies that specifically bind to NPA lectin or protein moieties.

Specific antibodies that can be used as the anti-NPA lectin-binding glycoprotein antibodies for detecting hepatocellular carcinoma markers and determining hepatocellular carcinoma in the present invention are indicated in Table 2 below.

TABLE 2

| | Abbre- | | | | | | |
|---|---|---|---|---|---|---|---|
| | viation/ | | [symbol] | | Catalog | Species/ | |
| Antigen | Symbol | MW | Mono | Vendor | number | Class | Subclass |
| [symbol] | CFH | 150 kDa | P | Santa Cruz | sc-33156 | Rabbit | IgG |
| factor H | | | | | | | |
| [symbol] | FBN1 | 350 kDa | M | [symbol] | H00022 [symbol] | Mouse/ [symbol] | IgG [symbol] |
| [symbol] | FN | 220 kDa | P | Santa Cruz | sc-9068 | Rabbit | IgG |
| (H-300) | | | | | | | |
| Oxygen | ORP-150, | 150 kDa | P | R [symbol] D | AF5568 | Goat | |
| regulated | HYOU1 | | | | | | |
| protein | | | | | | | |
| Epidural | EGFR | 175 kDa | P | Cell | [symbol] | Rabbit | IgG |
| growth | | | | Signaling | | | |
| factor | | | | | | | |
| receptor | | | | | | | |
| Cathapsin D | CTSD | 44 kDa | P | [symbol] | AF1014 | Goat | IgG |
| [symbol] -associated | LAMP2 | 120 kDa | M | Santa Cruz | sc- [symbol] | Mouse/ | |
| membrane | | | | | | SCH4B4 | |
| protein 3 | | | | | | | |
| [symbol] | PSAP | 70 kDa | P | [symbol] group | 100013AP | Rabbit | |

[symbol]

indicates data missing or illegible when filed

### (2-3) Regarding the Lectin-IGOT-LC/MS Method

Herebelow, the specific procedure for the “Lectin-IGOT-LC/MS method” used in the present invention will be briefly explained.

### (1) Preparation of ¹⁸O-Labeled Peptide

Protein samples prepared respectively from the culture supernatants of two different hepatocellular carcinoma cell lines (HLF and HAK1A), and from cancerous portions and non-cancerous portions from lesion tissues of hepatocellular carcinoma patients were passed through columns to which NPA lectin was bound so as to collect NPA-binding glycoprotein groups, which were fragmented into peptides by a trypsin treatment, then passed through the NPA lectin column again to re-collect the NPA lectin-binding glycoprotein groups. The obtained candidate glycoproteins were treated with peptide-N-glycanase (glycopeptidase F, PNGase F) so as to remove the N-linked glycans and instead introduce ¹⁸O at the Asn to which the glycans were bound, thereby stable isotope labeling the peptides. (This provides experimental verification as to whether glycans were bound to the peptides, and reveals the Asn on the peptide sequence to which the glycans were bound.)

### (2) Identification of the Amino Acid Sequences and the Glycan-Binding Positions on the Labeled Peptides

The candidate glycopeptides labeled by the IGOT method were isolated by liquid column chromatography (LC), subjected to mass spectrometry (MS), and by using tandem mass spectrometry (MS/MS ion search method), their amino acid sequences were comprehensively determined, and the search application Mascot was used to identify the glycan-binding positions.

(3) Identification of Glycoproteins that are Highly Expressed in Hepatocellular Carcinoma Tissue Cancerous Portions

The obtained NPA lectin-binding peptide groups were respectively associated with glycoproteins in a database, and commercially available antibodies to the corresponding glycoproteins were used to identify, as multiple candidate glycoproteins, the glycoproteins that were highly expressed in the cancerous portions relative to the non-cancerous portions in any of the hepatocellular carcinoma cell lines (HLF and HAK1A) and the lesion tissues from hepatocellular carcinoma patients.

### (4) Determination of Hepatocellular Carcinoma Marker Glycoproteins

The multiple obtained glycoproteins that can serve as hepatocellular carcinoma marker candidates were verified for the property of binding to NPA by actually observing the presence or absence of a band signal to an appropriate mobility in a Western blot due to anti-marker candidate protein antibodies in the NPA-binding protein fractions obtained after NPA collection, and those for which the signal appeared were selected as hepatocellular carcinoma marker glycoproteins in the present invention. In some cases, an antibody for the Western blot could not be obtained, and in those cases, a lectin-antibody sandwich ELISA was performed using NPA lectin and an anti-marker candidate protein antibody, and those in which a signal that was significantly stronger than the background signal (with an S/N ratio of at least 2) were chosen as the hepatocellular carcinoma marker glycoproteins of the present invention.

### 3. Hepatocellular Carcinoma Marker Detection Method of the Present Invention

### (3-1) Detection and Quantitative Determination by Lectin Array or Sandwich ELISA

The “NPA lectin-binding glycoproteins” that serve as the hepatocellular carcinoma marker in the present invention can be conveniently and accurately detected by lectin array or sandwich ELISA using NPA lectin, even when focusing only on the glycan moieties, and furthermore allows for quantitative determination of the hepatocellular carcinoma marker. The detection precision can be increased by using, in combination with NPA lectin, at least one lectin chosen from among LCA lectin, which is a fucose α1→6 glycan-binding lectin, ConA lectin, which is a high-mannose glycan-binding lectin that binds glycans having five or more mannoses, and SNA, SSA, TJAI and PSL1a lectins, which are α-2,6-sialic acid-binding lectins.

Additionally, hepatocellular carcinoma markers may be detected and quantitatively determined by using antibodies that recognize the protein moieties of NPA lectin-binding glycoproteins (e.g., anti-LAMP2 antibody, anti-CTSD antibody, anti-CFH antibody and anti-FBN1 antibody), or antibodies that simultaneously recognize the glycan and protein moieties. These anti-NPA lectin-binding glycoproteins may be used alone, but a sandwich ELISA method combined with lectins including NPA lectin is particularly preferred.

The methods for detection and quantitative determination of the hepatocellular carcinoma marker of the present invention can be used to determine whether or not a subject has developed hepatocellular carcinoma by detecting the hepatocellular carcinoma marker in a sample collected from the subject.

Additionally, by determining the amount of the hepatocellular carcinoma marker in serum (body fluid) collected after administering drugs for treating hepatocellular carcinoma, it is possible to assess the efficacy of the hepatocellular carcinoma treatment. For example, the hepatocellular carcinoma marker content or a value calculated therefrom can be determined before administering the therapeutic drug and at a point in time from days to months after administration and then compared, and if the hepatocellular carcinoma marker content or the value calculated therefrom is lower in the latter case, then it is possible to determine that there has been a preventive or therapeutic effect. Drugs for treating hepatocellular carcinoma include, for example, sorafenib (generic name).

In the present specification, “subject” refers to a person who is being tested, in other words, the person providing the test sample. The subject may be a patient suffering from some kind of disease, or may be a healthy individual. Preferably, the subject is a person who may be suffering from hepatocellular carcinoma, or a hepatocellular carcinoma patient.

The test sample may be a tissue fragment from a portion of hepatic tissue collected from the subject in a biopsy, or a tissue fragment from a lesion part of hepatic tissue resected from a hepatitis or cirrhosis patient. The subject is not particularly limited, and the determination of the presence of hepatocellular carcinoma can be widely applied to anyone in need thereof.

Additionally, a body fluid such as blood, lymph, spinal fluid or bile from the subject may be used, and it is most preferable to use serum obtained by separating blood collected from the subject as the test sample, since this does not place much of a burden on the subject and can shorten the testing time.

The analyte solution may be used immediately after being collected, or may be stored for a certain period of time in a freezer or a refrigerator, then used after treating by defrosting or the like as needed. In the present embodiment, in the case where serum is used, a sufficient quantity of the hepatocellular carcinoma marker can be detected by using a volume of 10 μL to 100 μL, 20 μL to 80 μL, 30 μL to 70 μL, 40 μL to 60 μL or 45 μL to 55 μL.

If a hepatocellular carcinoma marker is detected from the test sample by one of the below-described methods using a mannose-containing glycan-binding NPA lectin alone or preferably in combination with a hepatocellular carcinoma marker-detecting antibody, then it can be determined that the subject is suffering from hepatocellular carcinoma, or that there is a very high probability thereof.

### (3-2) Lectin Array Analysis of Tissue Fragments

When using a tissue fragment from hepatic tissue of the subject as the test sample, lectin array analysis can be performed, for example, using the following procedure.

The basic protocol followed in the present example is the technique of Matsuda et al. (Non-patent Document 10), and the following description will also primarily be based on that technique, but the invention is not limited thereto.

### <Preparation of Test Sample>

The tissue fragment is crushed in a buffer solution, the membrane proteins are solubilized, and the solution is centrifuged to obtain tissue-extracted proteins as the supernatant, and all of the tissue-extracted proteins are labeled.

As an alternative method, a labeled anti-NPA lectin-binding glycoprotein antibody obtained by fluoresecent-labeling an anti-NPA lectin-binding glycoprotein antibody that binds to the NPA lectin-binding glycoprotein which is the hepatocellular carcinoma marker may be used, but in that case, the tissue-extracted protein labeling step is unnecessary.

### <Labeling>

Examples of labeling substances include fluorescent substances (e.g. FITC, rhodamine, Cy3 and Cy5), radioactive substances (e.g. ¹⁴C and ³H), and enzymes (e.g. alkaline phosphatase, peroxidase (horseradish peroxidase, etc.) glucose oxidase and β-galactosidase). Additionally, binding between biotins and (strept)avidin may be used. A detection agent may be biotin-labeled, (strept)avidin may be labeled with a labeling substance, and detection may be performed by binding between biotin and (strept)avidin. The labeling methods mentioned here can be used for generally labeling the lectins used in the present invention, and furthermore, can be used for labeling the antibodies used in the present invention, such as anti-NPA lectin-binding glycoprotein antibodies that bind to NPA lectin-binding glycoproteins.

As the lectin array analysis, it is preferable to bind biotinylated NPA lectin to a solid phase coated with streptavidin, and to observe the binding to tissue-extracted proteins labeled with Cy3 or the like.

An enzyme can be used as the labeling substance, and detection is performed using a substrate that is appropriate for the enzyme being used. For example, when using peroxidase as the enzyme, o-phenylenediamine (OPD), tetramethylbenzidine (TMB) or the like is used as the substrate, and when alkali phosphatase is used as the enzyme, p-nitrophenyl phosphate (PNPP) is used as the substrate. The enzyme reaction stop solution and the substrate solution may also be appropriately chosen from among those that are well known, in accordance with the chosen enzyme.

Aside therefrom, a method of fluorescent labeling with 2-aminopyridine (PA) can be used when labeling the glycans, or a method of radiolabeling with a tritium label can also be used.

### <Preparation of Lectin Array>

The lectin array may be any kind of lectin array as long as it contains NPA lectin. For example, it is possible to use a lectin array having 45 plant lectins having different specificities immobilized on the same substrate, which was developed by the present inventors (Kuno et al., Nature Methods 2, 851-856, 2005) or LecChip™ Ver. 1.0 (manufactured by GlycoTechnica Ltd.), but the lectin array may be appropriately prepared in accordance with any known method.

The lectin array may use NPA lectin alone, but preferably has other lectins immobilized on a support. Examples of other lectins in this case include LCA lectin, ConA lectin, HPA lectin, DSA lectin, PHAL lectin, SNA lectin, SSA lectin, TJAI lectin, PSL1a lectin, UDA lectin, MAH lectin, GNA lectin, PWN lectin, UEAI lectin, MAL lectin, Calsepa lectin, ADL lectin, ACG lectin, PSA lectin and AAL lectin. The lectin array preferably includes LCA lectin, ConA lectin, HPA lectin, DSA lectin, SNA lectin and SSA lectin, among which LCA lectin and ConA lectin are particularly preferred.

While NPA lectin may be immobilized directly on the support (direct method), by using a biotinylated NPA as the NPA lectin and preparing the NPA lectin in a form immobilized on a streptavidin-coated support (indirect method), the detection sensitivity can be improved and the reduction in the background can be largely enhanced.

The support for the lectin array is preferably a transparent substance capable of transmitting evanescent waves, and stained glass or synthetic resins such as polycarbonates are commonly used.

### <Addition to and Washing of Lectin Array>

A tissue-extracted protein labeled with Cy3 or the like, diluted with a buffer solution or undiluted, is added to a lectin array reaction vessel and allowed to interact, after which non-specifically bound contaminants are washed away with a lectin array buffer solution (commercially available).

### <Detection Method>

The binding between lectins and glycans is generally weaker than binding with antibodies, such that the binding constant for antigen-antibody reactions is about 10⁶ to 10⁹ M⁻¹, while the binding constant between lectins and glycans is about 10⁴ to 10⁷ M⁻¹. In the case of the NPA lectin used in the present invention, even if the hepatocellular carcinoma marker is said to have strong binding, it is only of about the same level as normal lectins, so the signal should preferably be detected using evanescent wave-excited fluorescent detection. Evanescent wave-excited fluorescent detection is a method that makes use of the phenomenon wherein, when there are two phases with difference refractive indices such as glass (solid phase) and water (liquid phase), extremely short-range light (known as near-field light) known as evanescent waves seep out into the near field of about a few hundred nanometers from the interface. Using this method, when excitation light is made incident onto the end surface of a fluorescent substance, only the fluorescent substance present in the near field is excited in order to observe the fluorescence. Evanescent wave-excited fluorescent detection is described in Kuno et al., Nature Methods, 2, 851-856 (2005) and the like. The detection can be made by using a GlycoStation™ Reader 1200 (GlycoTechnica Ltd.).

Additionally, a similar detection method may be applied when using a labeled anti-NPA lectin-binding glycoprotein antibody.

### <Evaluation Method>

The evaluation by lectin array is performed by using a lectin for which the signal does not vary as a result of pathological changes, immobilized on the same lectin array substrate, as an internal control lectin, and relativizing the NPA signal, then determining whether or not a certain cutoff value has been exceeded. This method of relativizing the target lectin signal by using the value of a certain lectin as a control in order to make the determination has already been made public in a paper by the present inventors and is in the public domain, and reference should be made thereto (Kuno, A., et al, Clin. Chem., 2011 January, 57(1):48-56). The cutoff value may be set beforehand using collections of hepatic tissue specimens from a plurality of hepatocellular carcinoma patients. In other words, a discrimination formula is prepared on the basis of the above-mentioned relative value obtained by lectin array analysis using hepatocellular carcinoma parts and non-cancerous portions of hepatic tissues previously resected from a plurality of hepatocellular carcinoma patients. More preferably, a plurality of discrimination formulas corresponding to levels of progression or malignancy of the hepatocellular carcinoma are prepared, and the level of progression or malignancy of the hepatocellular carcinoma in a test sample is determined in order to determine the presence of hepatocellular carcinoma in a subject or the stage of advancement of the hepatocellular carcinoma.

### (3-3) Lectin-Antibody Sandwich ELISA

When using a tissue fragment from the hepatic tissue of a subject as the test sample, sandwich ELISA analysis may be performed, for example, using the following procedure.

The test sample preparation method, including labeling, is the same as in the lectin array analysis method described in (2-1). Next, a biotinylated NPA lectin is bound, for example, to a streptavidin-coated support, Cy3-labeled tissue-extracted proteins are added, and allowed to interact. Next, the support is washed with buffer solution, or left unwashed while blocking the unreacted NPA lectin, then reacted with an antibody (anti-Cy3/Cy5 antibody) that recognizes the Cy3 label.

It is also possible to apply a sandwich method using a labeled anti-NPA lectin-binding glycoprotein antibody obtained by labeling an anti-NPA lectin-binding glycoprotein antibody that is capable of recognizing and binding protein moieties (or glycans and protein moieties) of the NPA lectin-binding glycoproteins which are the hepatocellular carcinoma markers, without labeling the test tissue-extracted protein sample.

Additionally, the case is similar to that for lectin array analysis in that it is preferable to use NPA lectin in conjunction with other lectins such as LCA lectin, ConA lectin, HPA lectin and DSA lectin in a lectin array.

Furthermore, instead of a lectin array, it is possible to produce an antibody array having the anti-NPA lectin-binding glycoprotein antibodies immobilized on a support. In that case, after overlaying the test tissue-extracted protein sample, then performing detection by using labeled NPA lectin. In that case, detection may be performed on the test tissue-extracted protein sample by using an avidinated or biotinylated NPA lectin.

The results of the lectin-antibody sandwich ELISA may be applied to automation using an automatic immunodetection device. The only point that needs to be considered is the reaction between the antibody and the lectin used for sandwiching. The antibody has at least two N-linked glycans. Therefore, when the lectin being used recognizes a glycan on an antibody, background noise is caused by the binding reaction at the time of sandwich detection. In order to suppress the generation of this noise signal, it is possible to consider a method of introducing a modification on a glycan moiety on the antibody or a method of using only a Fab that does not include a glycan moiety, and known techniques may be used for this purpose. Methods for modifying the glycan moiety include, for example, those described in Chen, S., et al., Nat. Methods, 4, 437-44 (2007) and Comunale, M. A. et al., J. Proteome Res., 8, 595-602 (2009), etc., and methods using a Fab include, for example, that described in Matsumoto, H. et al., Clin. Chem. Lab. Med., 48, 505-512 (2010), etc.

### <Hepatocellular Carcinoma Marker Detection and Discrimination Method>

ELISA is a well-known technique, for which the optimal measurement device may be applied for each label, in accordance with the normal procedures.

The quantitative detection of the hepatocellular carcinoma marker according to the present method may be performed by using a protein that binds to NPA as a control substance to prepare calibrations lines, and converting them to equivalent quantities of the control substance. For example, a culture supernatant or a cell lysate of LecI cells, which are NPA-positive CHO mutant cells, as described in Example 2 (2-5), may be used as the control substance. When an NPA-positive cell is transfected with a gene for one protein which is then expressed to produce large quantities, it can be used as a more stable control substance. Additionally, as an evaluation method that does not use control substances, in accordance with the above-mentioned lectin array method, a lectin for which the signal does not vary with pathological changes may be used as an internal control lectin to relativize the NPA signal, and the determination can be made depending on whether or not a certain cutoff value is exceeded. The selection of the internal control lectin and the setting of the cutoff value can be performed beforehand by using collected hepatic tissue specimens from a plurality of hepatocellular carcinoma patients. In other words, the internal control may be statistically set beforehand by performing lectin array analysis on hepatocellular carcinoma parts and non-cancerous portions of hepatic tissues previously resected from a plurality of hepatocellular carcinoma patients. Additionally, a discrimination formula is prepared on the basis of the above-mentioned relative value obtained by ELISA measurements using hepatocellular carcinoma parts and non-cancerous portions of hepatic tissues previously resected from a plurality of hepatocellular carcinoma patients. More preferably, a plurality of discrimination formulas corresponding to levels of progression or malignancy of the hepatocellular carcinoma are prepared, and the level of progression or malignancy of the hepatocellular carcinoma in a test sample is determined in order to determine the presence of hepatocellular carcinoma in a subject or the stage of progression of the hepatocellular carcinoma.

### (3-4) Detection Method Using Tissue Staining

Additionally, since the NPA lectin-binding glycoproteins that serve as the hepatocellular carcinoma marker of the present invention, when considering the results of tissue staining and the like, are glycoproteins that are confined to the cell membrane surfaces of hepatocellular carcinoma and to immune cell membranes in regions in the vicinity of the cancer cells (TME), tissue staining can also be favorably used.

In other words, a portion of hepatic tissue collected from a subject by a biopsy or the like is sectioned, and NPA staining is performed using a labeled NPA lectin. Alternatively, an antibody that recognizes a hepatocellular carcinoma marker or other lectin may be used in conjunction therewith, and a sandwich method overlaying such an antibody or lectin may be used.

### (3-5) Method for Detecting Hepatocellular Carcinoma Marker in Test Serum Sample

Using the hepatocellular carcinoma detection method of the present invention, when performing early detection of hepatocellular carcinoma, a body fluid such as serum of the subject can be used as the test sample for detecting the hepatocellular carcinoma. Serum is the most preferable because it does not place much of a burden on the subject and can shorten the testing time. The hepatocellular carcinoma marker can be detected in the test sample by the detection method of the present invention, so as to detect and determine hepatocellular carcinoma originating in the liver at an early stage.

Even if the test sample is a body fluid such as serum, a lectin array analysis method and an ELISA analysis method can be applied in a manner similar to the case of a tissue specimen. In particular, it is preferable to apply the sandwich method described below.

In the sandwich method, it is preferable to use NPA lectin together with a substance that specifically binds to the protein moiety of an NPA lectin-binding glycoprotein, and as such a substance that binds to a protein moiety, it is preferable to use an anti-NPA lectin-binding glycoprotein antibody as described above.

As a specific method, the anti-NPA lectin-binding glycoprotein antibody is immobilized on a support, the NPA lectin-binding glycoprotein which is the hepatocellular carcinoma marker is prepared in a sandwiched form, the test sample is overlaid, and then detection can be performed by the labeled NPA lectin.

As another method, instead of immobilizing the antibody on the support, the NPA lectin-binding glycoprotein which is the hepatocellular carcinoma marker is presented on a reaction field having a plurality of lectins including NPA lectin immobilized on a support, and the labeled antibody is made to act on the overlaid test sample.

In the method of detection wherein a plurality of lectins including NPA lectin is immobilized on a support, an NPA lectin-binding glycoprotein is presented, and a labeled antibody is used, NPA lectin can be immobilized directly on the support (direct method), but as an improvement on this method, by using a biotinylated NPA as the NPA lectin and preparing the NPA lectin in a form immobilized on a streptavidin-coated support (indirect method), the detection sensitivity can be improved and the reduction in the background can be largely enhanced.

When using a sandwich method to measure the NPA lectin-binding glycoprotein of the present invention, the measurement may be performed by using ELISA, immunochromatography, a radioimmunoassay (RIA), a fluorescent immunoassay (FIA), a chemiluminescent immunoassay, or evanescent wave analysis. These methods are known among those skilled in the art and any of these methods could be chosen. Additionally, these methods may be used in accordance with the normal procedures, and the settings for the actual reaction conditions and the like could be made within the range that is normally carried out by those skilled in the art. Of these, it is particularly preferable to use lectin-antibody sandwich ELISA using an antibody and a lectin respectively as the protein-binding substance and the glycan-binding substance. The specific procedure for lectin-antibody sandwich ELISA is the same as that described in (3-3) above.

Additionally, in order to raise the sensitivity of the sandwich ELISA measurement system using a combination of lectins and antibodies, a detection system using chemiluminescence (Chemiluminescent Enzyme Immunoassay; CLEIA) may be applied.

The NPA lectin-binding glycoprotein in the test serum (body fluid sample) forms a complex with an NPA lectin or an anti-NPA lectin-binding glycoprotein antibody on the support used as a capturing agent. By measuring a signal generated by applying a labeled NPA lectin or labeled antibody as a detection agent to this complex, the NPA lectin-binding glycoprotein in the test sample is detected and quantitatively determined. The signal may be measured by using an appropriate measuring device in accordance with the labeling substance being used.

### (3-6) Multistage Lectin-Using Method

As mentioned in (3-5), it is most preferable to use a serum sample as the test sample for testing for the presence of hepatocellular carcinoma in a subject using the hepatocellular carcinoma marker of the present invention.

However, serum normally contains large quantities of many diverse types of glycoproteins, and the amount of NPA-binding proteins in blood secreted from cancer cells can be expected to be much less than that of other blood proteins. Additionally, it has been experimentally verified that many proteins that bind to NPA are originally present in blood.

Therefore, in the present invention, multistage lectin-using methods (Tan et al., Molecular BioSystems 2014) that use a lectin reaction property other than the property of binding to NPA, i.e. the property of not binding to α-2,6-sialic acid, as a technique for efficiently detecting the NPA-binding protein that serves as the hepatocellular carcinoma marker from a serum sample were studied.

As a result, it was discovered that many of the glycoproteins binding to NPA that are present in serum also simultaneously include α-2,6-sialic acid. In other words, by removing large quantities of glycoproteins having α-2,6-sialic acid from the serum beforehand by means of an α-2,6-sialic acid-binding lectin (SNA, SSA, TJAI or PSL1a), the NPA-binding proteins can be effectively concentrated, thereby raising the detection efficiency of the hepatocellular carcinoma marker.

For example, the proteins in a serum sample can be comprehensively Cy3-labeled and reacted beforehand with biotinylated α-2,6-sialic acid-binding lectins bound to streptavidin-coated magnetic beads, and the residual solution that did not bind can be obtained and applied to the lectin array.

### EXAMPLES

Herebelow, the present invention will be explained in detail by referring to examples, but the present invention is not to be construed as being limited thereto.

Other terminology and concepts in the present invention are based on the meanings of the terminology as commonly used in the relevant field, and the various techniques used to carry out the present invention can be easily and reliably carried out by those skilled in the art on the basis of publicly known documents or the like, with the particular exception of technologies that are expressly cited. Additionally, the various analyses were performed by referring to the methods described in the instruction manuals or catalogs for the analysis equipment or reagents and kits that were used.

The subject matter described in the technical documents, patent publications and specifications of patent applications cited in the present specification should be referred to as the subject matter of the present invention.

### (Example 1) Lectin Array Analysis of Tissues

The lectin microarray used in the present study is a system having 45 plant lectins with different specificities immobilized on the same substrate, for analyzing interactions (binding) with glycans on glycoproteins that are being analyzed (Kuno et al., Nature Methods 2, 851-856, 2005). This system was used to try to identify the optimal lectins that have signals with a significantly high value in a quantitative measurement system for hepatocellular carcinoma tissue, and that are capable of specifically staining cancerous portions by tissue staining Formalin-fixed paraffin-embedded hepatic tissues from hepatocellular carcinoma patients were used in the present experiments. Standard regions of cancerous portions and non-tumor hepatic parenchyma (non-cancerous portions) were respectively recovered as tissue fragments by laser microdissection (LMD), followed by protein extraction and fluorescent labeling, after which lectin array analysis was performed. The basic protocol followed that of Matsuda et al. (Biochem. Biophys. Res. Commun. 370, 259-263, 2008).

(1-1) Recovery of Proteins from Tissue Sections

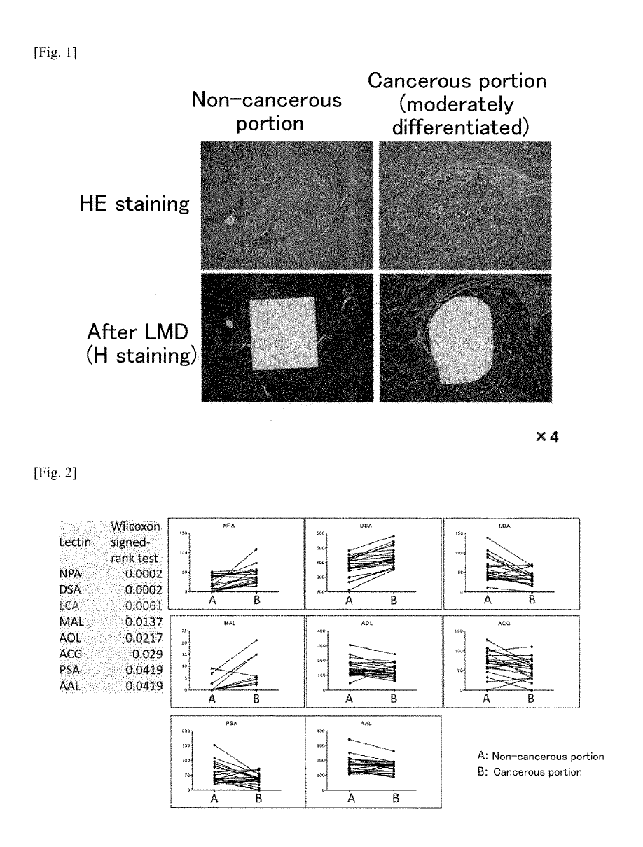

Tissue fragments were recovered from tissue sections using an LMD system, 6000DM (Leica Microsystems). Formalin-fixed tissue specimens for LMD were prepared by mounting pieces sectioned to a thickness of 5 μm on a film-coated glass (PEN-membrane, Leica Microsystems) which is a slide glass for use in LMD. While tissue specimens from hepatocellular carcinoma patients were used in the present experiment, the approval of the ethics committee was obtained at all facilities that were used. The tissue sections were nuclear-stained with hematoxylin to make them visible. Cancerous regions (equivalent to about 1×1 mm, see FIG. 1) from each sample were observed by microscope and cut away, and the tissue fragments were recovered in 0.6 mL tubes. In order to dissociate the intramolecular and intermolecular crosslinks due to the formalin, 200 μL of a 10 mM citrate buffer solution (pH 6.0) were first added to the obtained tissue fragments, and the solution was centrifuged (20,000 g, 1 min., 4° C.), and after confirming that the tissue fragments were in the buffer, treated at 95° C. for 60 minutes. After the heat treatment, the samples were centrifugally separated at 20,000×g for 1 minute at 4° C., and the supernatant was removed, after which 4 μL of a 50% slurry AVICEL suspension solution (AVICEL, manufactured by Sigma-Aldrich, suspended in MilliQ water and adjusted to a prescribed concentration) were added, and the solution was lightly tapped. After centrifugation (20,000×g, 1 min., 4° C.), 190 μL of supernatant were removed, and 190 μL of a PBS (−) buffer solution were added to the remaining tissue fragment-containing pellet (buffer exchange step). After further centrifuging at 20,000×g for 1 minute at 4° C. and removing the supernatant, 10 μL of a 1.0% NP40-PBS buffer solution was added to the pellet (the final concentration NP40 was 0.5%). After atomizing the pellet by ultrasonic fragmentation, the solution was allowed to react for 60 minutes on ice, and the membrane proteins were solubilized. After the reaction the sample was centrifuged at 20,000×g for 1 minute at 4° C., and the supernatant was recovered as tissue-extracted proteins.

### (1-2) Fluorescent Labeling of Proteins

All of the recovered tissue-extracted protein solutions were added to Cy3-SE (manufactured by GE Healthcare) that was pre-divided into 10 μg portions in PCR tubes. After allowing to react for 1 hour at room temperature in the dark, 80 μL of a glycine-containing buffer solution was added in order to inactivate the active groups in the excess Cy3-SE, and the solutions were allowed to react for 2 hours at room temperature in the dark. The obtained solutions were considered to be fluorescent-labeled tissue section-derived protein solutions.

### (1-3) Lectin Array Analysis

The fluorescent-labeled tissue section-derived protein solutions were diluted two- to four-fold with a lectin array buffer solution, and 60 μL of each was added to a reaction vessel. LecChip™ Ver. 1.0 (GlycoTechnica) was used as the lectin array. After allowing the solution to interact overnight at 20° C., each reaction vessel was washed three times using the lectin array buffer solution, and scanned in accordance with a conventional method. When a signal was not obtained in the above-described analysis, the above-mentioned fluorescent-labeled tissue section-derived protein solutions were added to the lectin array without dilution and analyzed. The obtained scan data were processed according to a conventional method to quantify the signals as net intensities, then standardized in accordance with the technique of Kuno et al. (J. Proteom. Bioinform 1, 68-72 (2008)) for subsequent statistical analysis.

### (1-4) Statistical Analysis

All of the standardized data were used for a two-group comparison test between cancerous portions (moderately differentiated) and non-cancerous portions. The data were subjected to a significance test using a Wilcoxon signed-rank test, which is a two-group comparison test with correspondence, for each lectin, and used to select lectins exhibiting significant signal elevation in the cancerous portions.

(1-5) Comparative Analysis Results of Hepatic Tissue Specimens from HCV-Infected Hepatocellular Carcinoma Patients

Blocks of hepatic tissues surgically resected from cases of hepatocellular carcinoma among hepatitis C patients were limited to types (called intranodal nodular) of HE-infected tissue that include cancer of multiple differentiation types differing from an immunological perspective, and 23 examples, all of which included moderately differentiated hepatocellular carcinoma, were used in the experiment. Using LMD, regions classified as moderately differentiated hepatocellular carcinoma, cancerous portions of other differentiation type (high differentiation or low differentiation), and non-cancerous portions were cut out to a total area of 1 mm² (FIG. 1). 69 sets of data were obtained by lectin array analysis, among which 23 sets of data respectively of moderately differentiated cancerous portions and non-cancerous portions were used to perform a two-group comparison test. As a result, statistical significance (P<0.05) was observed in eight lectins, as shown in FIG. 2. In particular, a marked signal increase was confirmed (P=0.0002) in the cancerous portions compared to the non-cancerous portions for NPA and DSA. Very interestingly, significantly low values were exhibited in the cancerous portions for all four lectins (LCA, PSA, AOL and AAL) classified as fucose-recognizing lectins.

(1-6) Comparative Glycan Analysis Results for Hepatic Tissue Specimens from Non-HCV- and Non-HBV-Infected Hepatocellular Carcinoma Patients

In order to demonstrate that the above-described results are not due to progressions in fibrosis or reduced function in the liver, but rather due to the occurrence of cancer, similar experiments were performed using hepatic tissue specimens from eight hepatocellular carcinoma patients not having a history of infection by either HBV or HCV. Such cases are rare, and it is difficult to demonstrate statistical significance using analysis of just one location from each case, so 1 mm² each was cut from regions of multiple cancerous portions and non-cancerous portions (19 cancerous portions and 20 non-cancerous portions) of each sample, and these were used in subsequent experiments. Therefore, a Mann-Whitney U-test, which is a non-parametric test without correspondence, was used for the statistical analysis. As a result, as shown in FIG. 3, there were 14 lectins that exhibited a significance of P<0.05, among which NPA exhibited a prominently high value in cancerous portions (P=0.0002), and ConA exhibited a low value (P=0.0002).

### (1-7) NPA-Binding Glycan Epitopes

As mentioned above, NPA alone exhibited a significantly high value at cancerous portions for both experiments. The glycan-binding specificity of this lectin will be discussed. FIG. 4 shows results obtained from the LfDB database (http://jcggdb.jp/rcmg/glycodb/LectinSearch), which allows systematic viewing of the affinities of lectins, showing the top ten glycans that bind to NPA, among the glycans registered in the database. The glycan-binding specificity analysis for ConA was not available in the LfDB, so the data was obtained by referring to the literature (Ohyama, Y. et al., J. Biol. Chem., 1985 Jun. 10; 260(11):6882-7), in which the data are explicitly provided.

In addition thereto, the drawing also shows the top ten glycans that bind to LCA, which is representative of lectins that exhibit binding to core fucose, to which NPA is partially specific, according to the literature. According to these results, the signal differences between cancerous portions and non-cancerous portions for NPA and LCA were completely different, being positive and negative. Therefore, it was suggested that the high values exhibited by NPA at cancerous portions were not due to binding with core fucose. Furthermore, NPA also exhibited an inverse correlation with ConA in the experimental results for non-B and non-C cases, thus suggesting that the values were not due to binding with high-mannose type glycans having more than five mannose units.

### (Example 2) NPA Lectin Reactivity Study of Cultured Hepatocellular Carcinoma Cells and Hepatocellular Carcinoma Patient Tissues by Sandwich ELISA

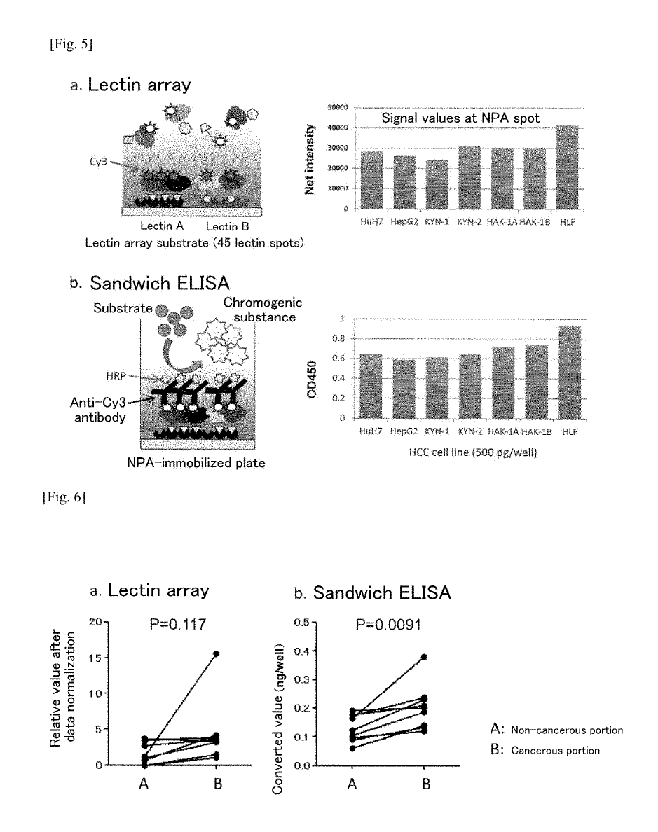

Using seven hepatocellular carcinoma culture cell lines (HuH-7, HepG2, KYN-1, KYN-2, HAK-1A, HAK-1B and HLF) that have been confirmed to be reactive to NPA by lectin array beforehand and hepatocellular carcinoma patient tissues, a study was carried out in order to determine whether the sandwich ELISA system shown in FIG. 5b could be constructed. The basic protocol from protein extraction from the cultured cells to fluorescent labeling followed the methods of Tateno et al. or Toyoda et al. (Methods Enzymol., 478, 181-195, 2010; Genes Cells, 16, 1-11, 2011). The samples were prepared from the tissue specimens in accordance with Example 1.

(2-1) Extraction of Proteins from Cultured Cells

The hepatocellular carcinoma cell lines were each prepared so as to contain 2×10⁶ per 1.5 mL tube. After pellet formation, the excess culture medium components and serum components were removed by washing three times with 1 mL of PBS (−). In the present experiment, in order to match with the method of protein extraction from the tissue sections, 200 μL of a 10 mM citrate buffer solution (pH 6.0) were added to the pellet, and the solution was treated for 90 minutes at 95° C. After the heat treatment, the solution was centrifuged at 20,000×g for 5 minutes at 4° C., and the supernatant was removed. 200 μL of PBS (−) were added to the remaining cell pellet (buffer exchange step). After further centrifuging at 20,000×g for 5 minutes at 4° C. and removing the supernatant, 40 μL of a 0.5% NP40-PBS buffer solution was added to the pellet. After atomizing the pellet by ultrasonic fragmentation, the solution was allowed to react for 60 minutes on ice, and the membrane proteins were solubilized. After the reaction, the sample was centrifuged at 20,000×g for 5 minutes at 4° C., and the supernatant was recovered as the tissue-extracted proteins.

### (2-2) Fluorescent Labeling of the Proteins

For the prepared cell-extracted protein solutions, the protein concentration of each of the culture cell protein extraction solutions was first measured by BCA. The BCA measurement was performed by using a MicroBCA kit (manufactured by Thermo Fisher Scientific), in accordance with the attached manual After the protein concentration measurement, 200 ng of each cell line-extracted protein were added to Cy3-SE (manufactured by GE Healthcare) that was pre-divided into 10 μg portions in PCR tubes. After allowing to chemically react for 1 hour at room temperature in the dark, 180 μL of a glycine-containing buffer were added in order to completely stop the reactions, and the solutions were allowed to react for 2 hours at room temperature in the dark. The obtained solutions were considered to be fluorescent-labeled tissue section-derived protein solutions.

### (2-3) NPA Lectin-Anti-Cy3 Antibody Sandwich ELISA

A microtiter plate (streptavidin-coated 96-well plate (NUNC Immobilizer)) was pre-washed twice with a washing solution (PBS containing 0.1% Tween 20), 50 μL of biotinylated NPL (manufactured by Vector Laboratories, 5 μg/mL) dissolved in PBS buffer solution were added to each well, and the plate was kept overnight at 4° C. to immobilize the NPL to the support. The unbound WFA was washed twice with a washing solution, to obtain an NPL-immobilized well plate. Next, Cy3-labeled protein solutions were adjusted to an amount of 50 μL in washing solution, added to the NPL-immobilized wells, and allowed to undergo a binding reaction for 1 hour at 37° C. After the reaction, 4 μL of a blocking agent (an asialofetuin solution adjusted to 0.5 mg/mL) were added to each well, and the solutions were allowed to react for 15 minutes at 37° C., so as to block the unreacted NPL lectin. After washing five times with washing solution to remove the unbound proteins, 50 μL of a detecting agent (anti-Cy3/Cy5 antibody, Sigma Aldrich) pre-adjusted to 0.125 μg/mL were added to each well, and antigen-antibody reactions were allowed to progress for 30 minutes at 37° C. After washing five times with washing solution in order to remove the unbound antibodies, 50 μL of an anti-murine IgG antibody-HRP solution (manufactured by Vector Laboratories) diluted 10,000-fold were added to each well, and maintained at a temperature of 37° C. for 20 minutes. After washing each well five times with washing solution, 100 μL each of 1-Step™ ULTRA TMB-ELISA Substrate Solution (manufactured by Thermo Fisher Scientific), a chromogenic reagent, were added to each well, and coloration reactions were allowed to progress for 30 minutes at room temperature. The reactions were then stopped by adding 100 μL of 1M H₂SO₄ solution to each well, and the absorbance at 450 nm was measured using a plate reader (SpectraMax M5, Molecular Devices). The plate was washed by adding 300 μL of washing solution to each well using a plate washer (ImmunoWash™ 1575 microplate washer, Bio-Rad Laboratories).

### (2-4) Sandwich ELISA Using Cells

From the cell lysates prepared from each of the seven hepatocellular carcinoma culture cell lines (HuH-7, HepG2, KYN-1, KYN-2, HAK-1A, HAK-1B and HLF), 200 ng by protein amount were labeled with Cy3, and the equivalent of 500 pg thereof were diluted with 50 microliters of a diluent and added to the wells. In order to raise the general applicability of the ELISA, chromogenic detection was used instead of fluorescent detection. As a result, the numerical values shown in FIG. 5b were obtained for each cell line. In order to investigate whether there is any correlation between the NPA signals in the lectin array analysis and the results of the present experiment, the remaining Cy3-labeled protein solutions were used to perform lectin array analysis. The results are shown in Table 5a. Comparing the measurement values for NPA lectin-anti-Cy3 antibody sandwich ELISA with the intensities of the NPA signals in a lectin array, it is apparent that there are similar tendencies in the relative intensity differences between the cells. The above-mentioned results suggest that the tendencies of NPA signals in which a significant difference was confirmed by comparative analysis by lectin array of cancerous portions and non-cancerous portions of hepatic tissue lysates of hepatocellular carcinoma patients as seen in Example 1 can be reproduced by NPA lectin-anti-Cy3 antibody sandwich ELISA measurements, which are simpler. Next, as a validation experiment thereof, the present experiment was performed using the cell lysates used in the lectin array analysis of Example 1.

### (2-5) Sandwich ELISA Using Tissues

From the tissue lysates for the 23 cases that have already been Cy3-labeled in Example 1, nine examples were randomly selected from among the cases in which enough excess solution existed to carry out NPA lectin-anti-Cy3 antibody sandwich ELISA measurements, and the NPA lectin-anti-Cy3 antibody sandwich ELISA measurements were performed using Cy3-labeled samples of lysates from the cancer portions and the non-cancer portions (for a total of 18 samples). 10 μL of each Cy3-labeled tissue lysate were adjusted to 50 μL using a washing solution, then added to respective wells. Using a cell extraction solution of a mutant CHO cell line (LecI), which is an NPA-positive cell, as a control glycoprotein solution, a two-fold dilution series was subjected to NPA lectin-anti-Cy3 antibody sandwich ELISA at the same time as the samples, and calibration lines were prepared therefrom. The measurement values for each sample were determined as conversion values to standard protein amounts using the calibration lines, and comparatively analyzed. The results are shown in FIG. 6. The cancerous portions exhibited significantly high values (P=0.0091) in NPA lectin-anti-Cy3 antibody sandwich ELISA also. The P value was determined by a Wilcoxon signed-rank test.

### (Example 3) Study of NPA Staining by Tissue Staining

### (3-1) Tissue Staining Method

Example 1 showed that it is possible to detect hepatocellular carcinoma in tissue sections by tissue staining using NPA. In order to validate the signal strength differences obtained for the results of the lectin array by tissue staining, the following study was performed using specimens serially pre-sectioned from the hepatic tissues of hepatocellular carcinoma patients when carrying out Example 1. The tissue specimens used for NPA staining were prepared from formalin-fixed paraffin-embedded blocks of hepatocellular carcinoma lesions including background liver disease collected at the gastrointestinal/general surgery department of the Kyushu University Graduate School. The paraffin was removed from tissue sections that were serially sectioned to a thickness of 5 μm, after which the tissue sections were treated for 10 minutes at 110° C. using REAL Retrieval Solution pH 6.0 (Dako) in order to activate the tissue sections. Next, Carbo-Free Blocking Solution (Vector Laboratories) was used to carry out a blocking treatment for 30 minutes at 20° C., and after washing three times in PBS, biotin-labeled NPL (Vector Laboratories) diluted to 5 μg/mL with 10 mM HEPES was added to the tissue sections, which were then allowed to react overnight at 4° C. After the reaction, the samples were washed three times in PBS, and allowed to react for 60 minutes at 20° C. with Alexa 488-labeled streptavidin (Life Technology), diluted to 20 μg/mL with PBS. After the reaction, the samples were washed three times in PBS, and allowed to react with hoechst33342 (Life Technologies) for 20 minutes at 20° C., to stain the nuclei. The NPA-specific signals were detected using a fluorescence microscope (KEYENCE).

### (3-2) Staining Results

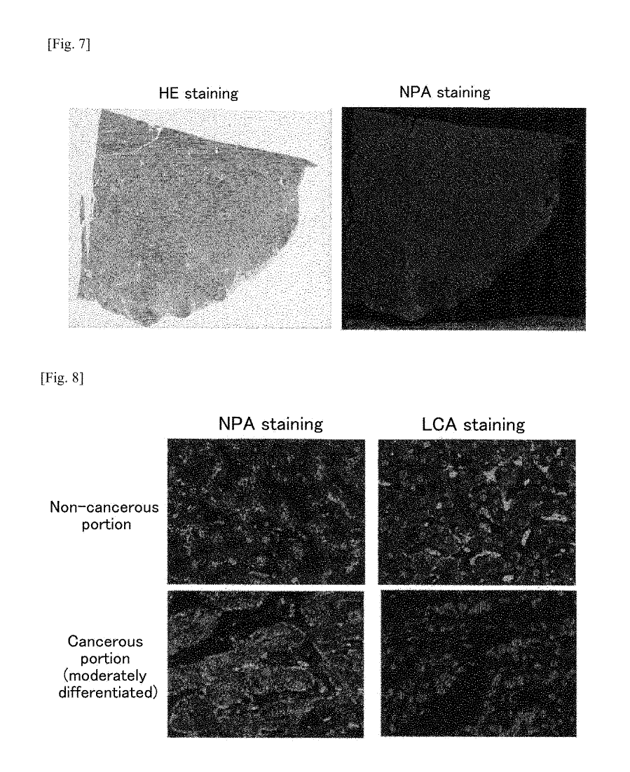

An image of one of the staining examples observed at a low magnification (wide field) is shown in FIG. 7. In the fluorescent staining images using NPA lectin, it appears at first glance as if the cancerous portions and the non-cancerous portions are stained uniformly. This tendency has been observed in other experiments using DAB staining, and in fact, in DAB staining the results indicated stronger staining in non-cancerous portions relative to cancerous portions.

Images of similar fluorescent-stained samples observed at high magnification (narrow field) are shown in FIG. 8. The observed locations correspond to the sites that were cut out by LMD during the lectin array analysis. Although there was no difference in the fact that areas emitting fluorescent light exist in both the cancerous portions and the non-cancerous portions, it was discovered, interestingly, that the staining pattern and intensity largely differed between the cancerous portions and the non-cancerous portions. In other words, in the non-cancerous portions, the hepatic parenchymal cells uniformly exhibited weak staining, and granular stains were contained inside the cells. In contrast, in the cancerous portions, granular stains were exhibited at the parts corresponding to the cell membranes and the parts located interstitially in the periphery of the cells, and the stain intensities thereof were relatively strong. In comparison thereto, the staining images for LCA lectin showed a pattern that largely differed from that of NPA staining, exhibiting strong staining in the carcinoma peripheral regions of the non-cancerous portions. These results reproduced those obtained by lectin array.

### (Example 4) Replication Test Using Tissues from Hepatocellular Carcinoma Patients

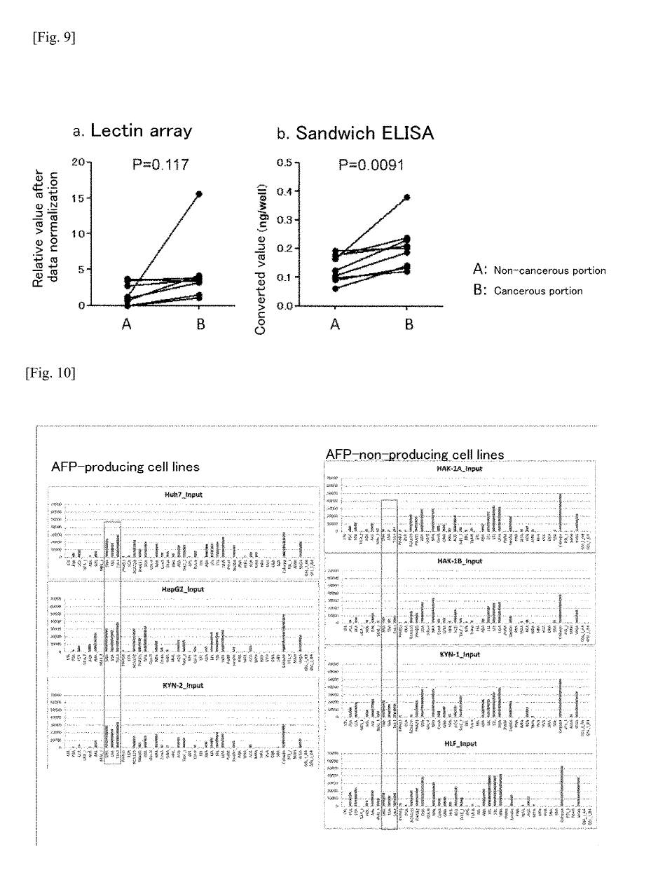

In order to demonstrate the validity of the experiments performed in the foregoing examples, replication tests were performed using tissues from hepatocellular carcinoma patients different from those in Examples 1-3. Formalin-fixed paraffin-embedded hepatocellular carcinoma tissue samples from seven cases of hepatocellular carcinoma patients, approved by the ethics committee, from the gastrointestinal/general surgery department of the Kyushu University Graduate School, were sectioned and affixed to slide glasses for laser microdissection (LMD). 49 parts were cut out in regions that were 1 mm square, from cancerous portions and non-cancerous portions respectively (for a total of 98 samples), and tissue lysates were prepared by the same method as that of Example 1 (1-1). These samples were subjected to lectin array analysis using the same method as in Example 1 (1-3) and NPA lectin-anti-Cy3 antibody sandwich ELISA analysis using the same method as in Example 2 (2-5) (FIG. 9).

As a result, significantly high values (p<0.01) were exhibited in the cancerous portions relative to the non-cancerous portions in both the lectin array analysis and sandwich ELISA analysis.

### (Example 5) Study of Other Types of Lectin Reactivity Characterizing NPA-Binding Proteins from Hepatocellular Carcinoma

Since blood that is secreted from hepatocellular carcinoma contains large quantities of blood proteins, the amount of NPA-binding proteins present can be expected to be much less than that of other blood proteins, even in serum from hepatocellular carcinoma patients. Additionally, it has been experimentally verified that blood originally contains proteins that bind to NPA. Since these can be expected to be a major cause of noise when the serum sample is used as a sample for detecting the hepatocellular carcinoma marker of the present invention, it is necessary to remove, as much as possible, the NPA-binding proteins that are not associated with hepatocellular carcinoma.

Therefore, in the present example, a multi-step lectin-using method making use of lectin arrays, proposed with reference to the method of Tan et al. (Molecular BioSystems, 2014), was performed in order to determine whether the characteristics of blood proteins secreted into the serum of hepatocellular carcinoma patients can be explained by reactivity to lectins other than the property of binding to NPA lectin.

Specifically, the Cy3-labeled secretory proteins prepared from the culture supernatants of seven hepatic carcinoma culture cell lines used in Example 2 and the Cy3-labeled tissue protein solution obtained in Example 2 (2-5) were each reacted with biotinylated NPA (selected NPA) (manufactured by Vector Laboratories) bound to magnetic beads pre-coated with streptavidin (manufactured by Veritas Corporation). The NPA-binding tissue proteins were recovered by a magnet, and the residual solution was applied to a lectin array. As a control, the same experiment was performed using magnetic beads not including lectins. After scanning, the characteristics of the NPA-binding proteins were extracted by numerical analysis.

The seven culture cell lines used in Example 2 can be largely divided between AFP-producing cell lines and AFP-non-producing cell lines depending on the difference in production of AFP (α-fetoprotein). Based on lectin array analysis of each type, it was discovered that there is a significant difference in reactivity to sialic acid between AFP-producing cell lines and AFP-non-producing cell lines, such that AFP-producing cell lines have relatively higher reactivity to α-2,6-sialic acid-recognizing lectins (FIG. 10). On the other hand, as with the experimental results of Example 2, all of the cell lines exhibited strong reactivity to NPA.

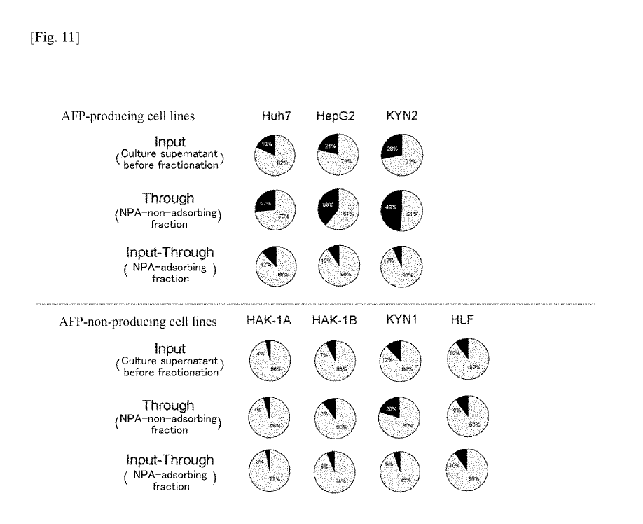

Next, by means of a multi-step lectin-using method, the NPA-binding glycoprotein group was adsorbed to the beads, and the supernatant of the non-adsorbed fraction (Through fraction) was applied to a lectin array, and after obtaining the data, the difference from the original data (Input) was obtained as the lectin array profile (Input-Through) of the NPA-binding glycoprotein group. As mentioned above, in the AFP-producing cell lines, the proportion of the signals of the α-2,6-sialic acid-recognizing lectin group was relatively higher, but upon investigating the proportion of signals of the α-2,6-sialic acid-recognizing group in the NPA-binding glycoprotein group (Input-Through in FIG. 11), the proportion was largely reduced, and became about the same as in the AFP-non-producing cell lines. That is, as a characteristic that is common to all hepatocellular carcinoma-derived cells, it became clear that there are NPA-binding glycoproteins that do not exhibit the property of binding to α-2,6-sialic acid-recognizing lectins. This tendency was also supported by experiments using Cy3-labeled tissue protein solutions.

This characteristic of not binding to α-2,6-sialic acid-recognizing lectins is demonstrated as being effective for enriching hepatocellular cancer-derived NPA-binding glycoproteins that are present in blood. In other words, as mentioned above, blood originally contains large quantities of glycoproteins that bind to NPA, and there is little significant difference between healthy individuals and carcinoma patients. Additionally, it has been experimentally shown that the majority of the glycoproteins exhibit the property of binding to α-2,6-sialic acid-recognizing lectins. Meanwhile, according to the results of the current experiments, hepatocellular carcinoma-derived cells all secrete NPA-binding glycoproteins that are not recognized by α-2,6-sialic acid. In view thereof, it can be inferred that hepatocellular carcinoma-derived NPA-binding proteins can be easily captured by first applying a test serum to an α-2,6-sialic acid-recognizing lectin column, adsorbing and removing proteins binding to α-2,6-sialic acid-recognizing lectins, and analyzing the NPA-binding glycoproteins in the non-adsorbed fraction.

### (Example 6) Enrichment of NPA-Binding Proteins in Serum of Non-B and Non-C Primary Hepatic Cancer Patients by the Multi-Step Lectin-Using Method

As indicated in Example 5, the serum of healthy individuals contains large quantities of glycoproteins that bind to NPA. However, most of those are known to bind also to α-2,6-sialic acid. Therefore, it was investigated whether there is a significant qualitative difference between healthy individuals and cancer patients in the protein groups collected by NPA after adsorbing and removing the α-2,6-sialic acid-containing glycoproteins from the serum in accordance with the multi-step lectin-using method. While most hepatic cancer occurs in those who have been infected by a virus, in such cases, fibrosis occurs in the background liver and this causes glycan changes, so even if there is a difference in comparison with healthy individuals, there is still the possibility of not being able to judge whether the difference is due to cancer or due to differences in the background liver (differences in the degree of fibrosis progression), so experiments were performed using serum from (non-B and non-C) primary hepatic cancer patients not having a history of HBV or HCV.

SSA lectin (manufactured by J-Oil Mills) was used as the α-2,6-sialic acid-recognizing lectin, SSA-immobilized beads were produced, and an SSA binding reaction was carried out. First, 10 μl of washed streptavidin beads were dispensed into 1.5 ml microtubes, 10 μl each of a lectin solution (containing 1 μg of biotinylated SSA, which is an α-2,6-sialic acid-recognizing lectin) were added, and these were mixed and reacted for 30 minutes at 4° C. After adsorbing the beads to a magnet, the supernatant was removed (this supernatant was identified as Through 1), and the remaining beads were washed three times with 1% Triton X-100-containing PBS (PBSTx). 10 μl (corresponding to 0.001 μl of serum) of a Cy3-labeled serum protein were added thereto, and the solution was mixed and reacted overnight at 4° C. After adsorbing the beads to a magnet, the supernatant was collected in a new tube as an SSA-non-adsorbing fraction (this was identified as Through 2), and used in a subsequent NPA binding reaction. The remaining beads were washed three times with PBSTx, then 10 μl of 0.2% SDS-containing PBS was added and mixed, then subjected to a heat treatment at 95° C. for 5 minutes, then cooled, after which the beads were adsorbed to the magnet and the supernatant was collected as an SSA-adsorbing fraction (this supernatant was identified as Elution 1).

In order to carry out the NPA binding reaction, 10 μl of washed SA beads were dispensed into 1.5 ml microtubes, 10 μl each of a lectin solution (containing 1 μg of biotinylated NPA) were added, and these were mixed and reacted for 30 minutes at 4° C. After adsorbing the beads to a magnet, the supernatant was removed (this supernatant was identified as Through 3), and the remaining beads were washed three times with PBSTx. The SSA-non-adsorbing fraction (Through 2) was added thereto, and the solution was mixed and reacted overnight at 4° C. After the reaction, the supernatant was collected in a new tube as an SSA-NPA-non-adsorbing fraction (this was identified as Through 4). The remaining beads were washed three times with PBSTx, then 10 μl of 0.2% SDS-containing PBS was added and mixed, then subjected to a heat treatment at 95° C. for 5 minutes. After cooling, the beads were adsorbed to the magnet and the supernatant was collected (this supernatant was identified as Elution 2). 10 μl of washed SA beads were added thereto, and the solution was mixed and reacted for 30 minutes at 4° C. After the reaction, the supernatant was collected in a new tube as an SSA-NPA-non-absorbing fraction (this was identified as Elution 3).

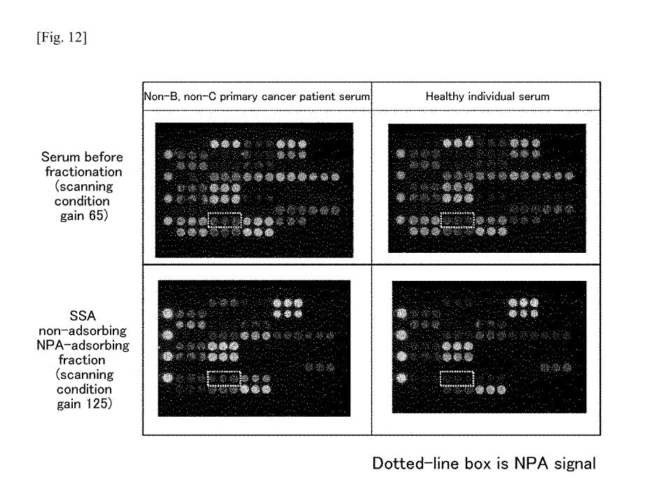

The above-described experiments were performed using serum from healthy individuals and serum from non-B and non-C primary hepatic cancer patients, and the respective fractions were subjected to lectin array analysis. Scan data for the serum before fractionation and the SSA-non-adsorbing NPA-adsorbing fraction are shown in FIG. 12.

As a result, in the serum before fractionation, there was no big difference in the profiles between the serums from cancer patients and from healthy individuals, and there was no significant difference in the NPA signals. This supports the theory that there are only tiny quantities of cancer-derived glycoproteins in blood. On the other hand, in the SSA-non-adsorbing NPA-adsorbing fraction, it was discovered that the signal is significantly higher in the serum of cancer patients for multiple lectins including NPA. This means that this fraction contains secreted glycoproteins from primary cancer.

### (Example 7) Identification of Hepatocellular Carcinoma Marker Candidate Glycoproteins by Glycoproteomics (IGOT-LC/MS)

In this example, the glycan peptide identification method using Lectin-IGOT-LC/MS, which was previously developed by the present inventors (Japanese Patent No. 4220257 etc.), is applied to glycoprotein samples from the culture supernatant of hepatocellular carcinoma culture cell lines and lesion tissues from hepatocellular carcinoma patients, in order to identify glycoproteins as hepatocellular carcinoma marker candidates.

(7-1) Preparation of Labeled Peptides by IGOT Using Samples from Human Hepatocellular Carcinoma Culture Cell Lines

Among the hepatocellular carcinoma culture cell lines used in Example 2, the HLF line and the HAK1A line were each cultured using a 10% FBS-containing culture medium, after which the culture solution was sucked out and discarded, a serum-free culture medium was newly added, washed four times, and the serum-free culture medium was added and the result cultured for 48 hours. The culture supernatant was collected and centrifuged for 30 minutes at 3100 rpm, after which the supernatant was recovered. The remaining cell pellet was also preserved for use in analysis. The supernatant was concentrated 30-fold using an ultrafiltration membrane having a 3K molecular weight cutoff, filtered using a 0.45 μm filter, and the proteins were precipitated by acetone precipitation. After recovering the precipitate, the pressure was reduced for a short time to remove the acetone, resulting in a culture medium protein concentrate (precipitate).

The resulting culture medium protein concentrate (precipitate) and the cells were solubilized using a guanidine solution according to a conventional method, then subjected to high-speed centrifugal separation, and the supernatant (extraction solution) was recovered. After removing dissolved oxygen by means of nitrogen gas, dithiothreitol (DTT) was added in an amount equal to the weight of the protein, in the form of a powder, or dissolved in a small amount of a solubilizing buffer solution.

The solution was allowed to react for 1 to 2 hours at room temperature in the presence of nitrogen gas in order to reduce the disulfide bonds. Next, for S-alkylation, 2.5-times the weight of the protein of iodoacetamide were added, and the solution was allowed to react for 1 to 2 hours at room temperature while blocking light. After the reaction, the solution was dialyzed with 50 to 100 times the amount of a buffer solution, to remove modifying agents (guanidine hydrochloride) and excess reagent. After quantitative determination of the proteins, 1/100 to 1/50 by weight of trypsin and 1/100 to 1/200 by weight of lysyl endopeptidase with respect to the amount of protein were added, and digestion was allowed to progress overnight (about 16 hours) at 37° C. Phenylmethanesulfonyl fluoride (PMSF) with a final concentration of 5 mM was added and the reaction was stopped. The digested product was subjected to hydrophilic interaction chromatography using an Amide 80 column, and the glycopeptide fraction was captured.

After diluting with a buffer solution (50 mM Tris hydrochloride buffer solution, pH 7.5), the solution was added to an NPA-agarose column equilibrated with the same buffer solution, and after washing, elution was performed using the same buffer solution containing 0.2 M methyl mannoside. The glycopeptide fraction was provided to an ODS column and the eluted sugars and salts were removed. A fraction eluted by 70% acetonitrile (0.1% TFA) was identified as the sample glycopeptide (NPA (+)). After drying, water labeled with the stable isotope oxygen-18 (H₂¹⁸O) and peptide-N-glycanase F were added to cleave the glycans and the glycosylation sites were labeled to prepare labeled peptide samples from the culture cell lines.

(7-2) Preparation of Labeled Peptides from Tissue Samples from Human Hepatocellular Carcinoma Patients

One of the formalin-fixed paraffin-embedded hepatocellular carcinoma tissues from hepatocellular carcinoma patients used in Example 4 was sectioned to a thickness of 5 μm, fixed to a slide glass for laser microdissection (LMD), and about 1.8 mm² each of cancerous portions and non-cancerous portions were cut out at multiple locations using an LMD.

Three sections of cancerous portions were allowed to swell in a PTS buffer solution (0.1 M Tris hydrochloride buffer solution, pH 9.0, containing 12 mM deoxycholic acid and 12 mM N-lauroylsarcosine sodium), ultrasonically treated, and heated for 1 hour at 100° C. The samples were reduced with dithiothreitol (DTT) in a nitrogen atmosphere, then alkylated with iodoacetamide. After dilution with 50 mM of ammonium hydrogen carbonate, pH 8.6, the samples were digested overnight (18 hours) at 37° C. using trypsin and lysyl endopeptidase. 1 mM of PMSF was added to the result, and the reaction was stopped. An equal amount of ethyl acetate was added, a surfactant was extracted and removed in the organic phase, and the peptides in the lower layer were recovered. These were subjected to hydrophilic interaction chromatography using an Amide 80 column (TOSOH), and the glycopeptides were captured. These were diluted with a buffer solution (50 mM Tris hydrochloride buffer solution, pH 7.5), an NPA-immobilized agarose gel was added thereto, and the solution was allowed to react for 30 minutes at room temperature. After recovering the supernatant by centrifugation, the beads were washed in the same buffer solution to remove the unreacted matter. After drying the beads, water labeled with the stable isotope oxygen-18 (H₂¹⁸O) and peptide-N-glycanase F were added to cleave the glycans and the glycosylation sites were labeled to prepare labeled peptide samples from patient tissues.

### (7-3) LC/MS Shotgun Analysis of Labeled Peptides

The labeled peptide samples from the culture cell lines and from patient tissues obtained in (7-1) and (7-2) were diluted with 0.1% formic acid, and subjected to an LC/MS shotgun analysis. An injected candidate glycopeptide was temporarily captured on a desalination trap column (reversed-phase C18 silica gel support), washed, then separated by an acetonitrile concentration gradient method using fritless microcolumns (inner diameter 150 μm×50-100 mm) in the form of spray tips packed with the same resin. The eluate was ionized by an electrospray interface, and directly introduced into a mass spectrometer. The mass spectrometry was performed using tandem mass spectroscopy by collision-induced dissociation (CID) while choosing a maximum of 10 ions in a data-dependent mode.

### (7-4) Search and Identification of Candidate Glycopeptides by MS/MS Ion Searching Method

The thousands of resulting MS/MS spectral data files were converted to a Mascot-generic file (mgf) using Proteome Discoverer (software available from Thermo Scientific). A protein amino acid sequence database was used to perform an MS/MS ion search based on this data, thereby identifying candidate glycoproteins.

Identification confirmation processes were performed on the basis of the fact that there are N-linked glycosylation consensus sequences in the identified peptides, and there are that number or fewer Asn modifications (conversions to Asp and ¹⁸O intake), to obtain hepatocellular carcinoma-discriminating marker glycopeptide candidates.

(7-5)

The “peptide sequences” of these hepatocellular carcinoma-discriminating marker glycopeptide candidates were matched with the amino acid sequences of full-length glycoproteins using the amino acid sequence database NCBI-Refseq. Among these glycoproteins, eight glycoproteins (EGFR, FN1, FBN1, HYOU1, CFH, PSAP, CTSD and LAMP-2) that were previously confirmed to be highly expressed in hepatocellular carcinoma cells were further studied as to whether or not they could serve as hepatocellular carcinoma marker candidates.

### (Example 8) Validation of Glycoproteins Serving as Hepatocellular Carcinoma Marker Candidates (Western Blot Analysis with NPA-Binding Fraction in Cell Extracts from Culture Cell Lines)

The present example further validates the significance of the hepatocellular carcinoma candidate glycoprotein molecule group chosen in (Example 7), and confirms that they are expressed as NPA-binding glycoproteins in hepatocellular carcinoma cell lines using cell extracts from hepatocellular carcinoma cell lines.

### (8-1) Fractionation of Test Samples by Lectin Affinity

Of the hepatocellular carcinoma cell lines used in Example 2, cell extracts were obtained from the Huh7, HAK 1A and HLF cell lines in accordance with the method described in Example 2. 1 μg of biotinylated NPA (Vector Laboratories) were added to 10 μL of streptavidin-fixed magnetic beads (Invitrogen) suspended in 1% Triton X-100-containing PBS (PBSTx), and the solution was mixed and reacted for 30 minutes at 4° C. to immobilize the biotinylated NPA on the magnetic beads. After adsorbing the beads to the magnets, the supernatant was removed, and the beads were washed three times with 200 μL of PB STx. After washing, 10 μg of each sample, by the total amount of protein, was adjusted to 100 μL using PBSTx, the above-mentioned beads were added thereto, and the solution was mixed and reacted overnight at 4° C. After adsorbing the beads to the magnet, the supernatant was removed, 10 μL of 0.2% SDS-containing PBS were added to the beads, and the adsorbed matter was eluted by performing a heat treatment for 10 minutes at 95° C. After ice-cooling for 1 minute, 10 μL of the supernatant was transferred to a new tube, the equivalent of 20 μL of streptavidin beads were added, the solution was adjusted to 20 μL using PBSTx, and the solution was mixed and reacted for 1 hour at 4° C. to remove the excess biotinylated NPA. After the reaction, the supernatant (20 μL) was recovered, and this was identified as an NPA-binding protein elution fraction.

(8-2) Detection of Hepatocellular Carcinoma Marker Molecules by Western Blot of Cell Lines from Hepatocellular Carcinoma

The obtained NPA-binding protein elution fractions were electrophoresed using a 10% polyacrylamide gel under SDS-PAGE reducing conditions, and transferred to a PVDF membrane. After blocking with PBS containing 5% skim milk, anti-HYOU1 antibody (manufactured by R&D Systems), anti-EGFR antibody (manufactured by Cell Signaling Technology), anti-PSAP antibody (manufactured by Proteintech Group), anti-CTSD antibody (manufactured by Life Span Biosciences) and anti-LAMP-2 antibody (Santa Cruz Antibody) were used to detect HYOU1, EGFR, PSAP, CTSD and LAMP-2 glycoprotein molecules by Western blot. The Western blot was performed by reacting with each of the above-mentioned primary antibodies for 1 hour at room temperature in accordance with a general method. After washing the PVDF membranes, they were reacted with a commercially available secondary antibody (0.5 μg/mL) such as anti-Goat IgG-HRP (manufactured by Jackson ImmunoResearch) for 1 hour at room temperature. After washing these PVDF membranes, detection was performed by chemiluminescence using a Western blot detection reagent (PerkinElmer).

### (Results)

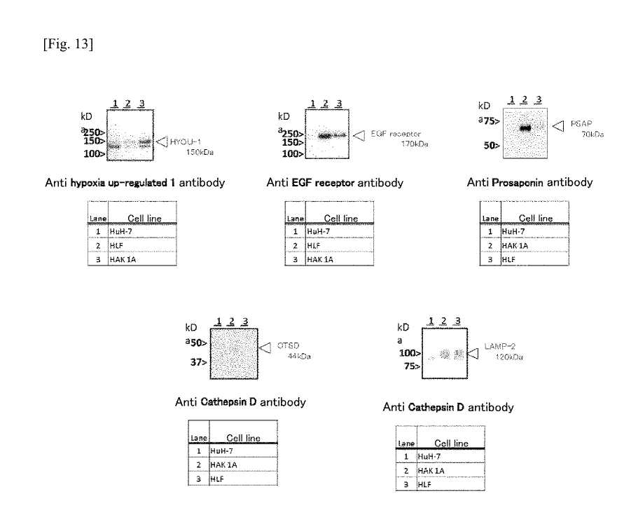

The results are shown in FIG. 13. Each marker molecule was detected from one of the NPA-binding fractions of the hepatocellular carcinoma-derived cell lines. As a result, it was verified that the HYOU1, EGFR, PSAP, CTSD and LAMP-2 glycoproteins of the present invention are all molecules that are expressed in hepatocellular carcinoma, and that have NPA-binding glycans.

### (Example 9) Validation of Glycoproteins Serving as Hepatocellular Carcinoma Marker Candidates (Western Blot Analysis with NPA-Binding Fraction in Culture Supernatants from Culture Cell Lines)

The present example further validates the significance of the CFH, FN1, PSAP, CTSD and LAMP-2 glycoproteins in the hepatocellular carcinoma candidate glycoprotein molecule group chosen in (Example 7), and confirms that they are expressed as NPA-binding glycoproteins in hepatocellular carcinoma cell lines using culture supernatants from hepatocellular carcinoma cell lines.

Serum-free culture supernatants of the Huh7, HAK 1A, HAK and HLF cell lines among the hepatocellular carcinoma cell lines used in Example 2 were fractionated with NPA lectin by the same method as in (8-1). Anti-CFH antibody (manufactured by Santa Cruz Biotechnology), anti-FN1 antibody (manufactured by Santa Cruz Biotechnology), anti-PSAP antibody (manufactured by Proteintech Group), anti-CTSD antibody (Life Span Biosciences) and anti-LAMP-2 antibody (Santa Cruz Biotechnology) were used to detect CFH, FN1, PSAP, CTSD and LAMP-2 glycoprotein molecules by Western blot.

These NPA-binding protein elution fractions were electrophoresed using a 10% polyacrylamide gel under SDS-PAGE reducing conditions, and transferred to a PVDF membrane. After blocking with PBS containing 5% skim milk, the PVDF film was reacted with the above-mentioned primary antibodies (CFH antibody and FN1 antibody) for 1 hour at room temperature. After washing the PVDF membranes, they were reacted with a commercially available secondary antibody (0.5 μg/mL) for 1 hour at room temperature. After washing these PVDF membranes, detection was performed by chemiluminescence using a Western blot detection reagent (PerkinElmer).

### (Results)

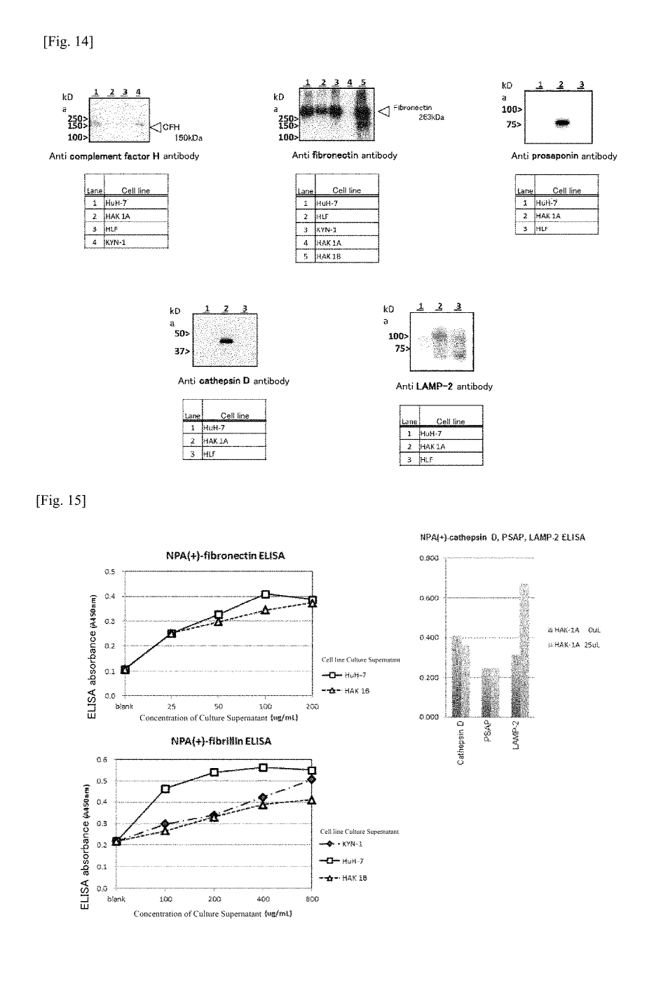

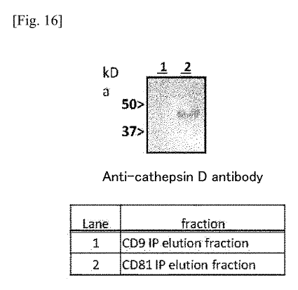

The results are shown in FIG. 14. Each marker molecule was detected from among the NPA-binding fractions of the culture supernatant of hepatocellular carcinoma cells. As a result, it was shown that the CFH, FN1, PSAP, CTSD and LAMP-2 glycoproteins of the present invention are all secreted glycoproteins having NPA-binding glycans.

### (Example 10) Verification of Glycoproteins Serving as Hepatocellular Carcinoma Marker Candidates (Detection of Marker Molecules by NPA Lectin-Antibody Sandwich ELISA Measurement System in Culture Supernatants of Culture Cell Lines)

The present example further validates the significance of the FBN1, FN1 and LAMP-2 glycoprotein molecules in the hepatocellular carcinoma candidate glycoprotein molecule group chosen in (Example 7), and confirms that they are expressed as NPA-binding glycoproteins in hepatocellular carcinoma cell lines using cell extracts from hepatocellular carcinoma cell lines.

### (10-1) Detection of Marker Molecules by NPA Lectin-Antibody Sandwich ELISA Measurement System-1

### (Method)

Among the hepatocellular carcinoma cell lines used in Example 2, NPA lectin fractionation was performed on the culture supernatants of serum-free cultures of the HuH-7, HAK 1B and KYN-1 cell lines, using the same method as in (8-1). Using the anti-FBN1 antibody (manufactured by Abnova) and an anti-FN1 antibody (manufactured by Santa Cruz Biotechnology), FBN1 and FN1 glycoprotein molecules were detected by an NPA lectin-antibody sandwich ELISA measurement system. The anti-FBN1 antibody and the anti-FN1 antibody were each used on the ELISA plate-immobilized side to perform an examination on the sandwich ELISA measurement system.

First, the anti-FBN1 antibody and the FN1 antibody were diluted with PBS to 4 μg/mL, and 100 μL/well were added to an ELISA microplate (Thermo Scientific Nunc 436013, Immobilizer [Amino] Plate). After adsorbing each antibody to the plate overnight at 4° C., the solution was discarded and the wells were washed with PBS-T (PBS, 0.05% Tween-20). Next, 300 μL/well of TBS (50 mM Tris, 150 mM NaCl, pH 8.0, 0.1% NaN₃) were added as a blocking solution, and blocking was performed. The blocking solution was discarded, and after washing, 100 μL of the solutions containing the samples (culture supernatants of serum-free cultures of the hepatic cancer cell lines HuH-7, HAK 1B and KYN-1) were added to each well. After reacting for 2 hours at room temperature, the solutions in the wells were discarded, and after washing with PBS-T, biotin-labeled NPA lectin was adjusted to 2 μg/mL in each well, and the solutions were allowed to react for 1.5 hours at room temperature. Thereafter, the solutions were discarded, and after washing, 100 μL of a horseradish peroxidase (HRP)-labeled streptavidin (Jackson ImmunoResearch) solution were added to each well, and allowed to react for 1 hour at room temperature. After discarding the reaction solution and rinsing, coloration by a 1StepUltra TMB substrate solution (Thermo Scientific) was measured by absorbance at 450 nm.

The reactivity of FBN1 and FN1 glycoproteins studied in the above-described examples were concentration-dependently confirmed by NPA-antibody sandwich ELISA. (It was confirmed that reactivity was not observed in a negative control using only a buffer). The results are shown in FIG. 15. These show that both the FBN1 and the FN1 glycoproteins of the present invention are secreted glycoproteins having NPA-binding glycans, and are secreted by hepatocellular carcinoma cells.

### (10-2) Detection of Marker Molecules by NPA Lectin-Antibody Sandwich ELISA Measurement System-2

As in (10-1), NPA lectin fractions from the culture supernatants of serum-free cultures of the hepatocellular carcinoma cell line HAK-1A were used to immobilize anti-CTSD antibody (manufactured by Life Span Biosciences), anti-PSAP antibody (manufactured by Proteintech Group) and anti-LAMP-2 antibody (Santa Cruz antibody) to ELISA plates and to perform sandwich ELISA analysis with NPA lectin.

As a result, in the case of the HAK-1A line among the hepatic cancer cells, the secretion of the CTSD and PSAP glycoproteins was no greater than the detection limit, but it was confirmed that at least the LAMP-2 glycoprotein is significantly secreted as a secretory glycoprotein having an NPA-binding glycan (FIG. 15).

### (Example 11) Detection of Hepatic Cancer Marker Molecules in Exosome Fractions

The present example verifies, as one possible reason why the specific presence of glycoproteins that are NPA-binding glycoproteins of the present invention and that are originally present in the membrane fraction or in lysosomes is confirmed in the TME in the vicinity of hepatocellular carcinoma cells, the possibility that the hepatocellular carcinoma cells are secreted as glycoproteins in exosomes. Recently, there have been many reports elucidating that exosomes are granules that are secreted by cancer cells, and that play an important role in the metastasis of cancer (Nat. Med., 2012 June; 18(6):883-91 doi:10.1038/nm.2753, etc.).

### (Method)

Anti-CD9 antibody (manufactured by Cosmo Bio) and CD81 antibody (manufactured by Cosmo Bio), antibodies to CD9 and CD81, which are exosome markers, were used to concentrate exosomes from the serum-free culture supernatant of hepatocellular carcinoma cell line HAK 1A by immunoprecipitation.

Specifically, 500 ng each of biotinylated anti-CD9 antibody and CD81 antibody were reacted for 1 hour at 4° C. with 10 μL of the streptavidin-coated magnetic beads used in (Example 8), to prepare biotinylated antibody-immobilized beads. The beads were washed three times with 200 μL of 0.1% Tween20-containing PBS (PBSt), after which 20 μg of HAK 1A culture supernatant was diluted to 20 μL using PBSt, then added to the beads, and an antigen-antibody reaction was allowed to take place overnight at 4° C. After removing the supernatant and washing the beads three times with 200 μL of PB St, 10 μL of 0.2% SDS-PBS were added to the beads and a heat treatment was applied for 10 minutes at 95° C. to cause elution of the bound glycoproteins. After ice-cooling for 1 minute, 10 μL of two-fold concentrated streptavidin beads were added to the supernatant and allowed to react for 1 hour at 4° C., thereby removing excess eluted biotinylated antibody, and the resulting supernatants were identified as the CD9- and CD81-binding fractions. These fractions were subjected to Western blotting of the molecules using anti-CTSD antibody (Life Span Biosciences). These were then electrophoresed with 10%-20% SDS-polyacrylamide gel and transferred to PVDF membrane. Using a blocking solution (Block Ace, manufactured by DS Pharma Biomedical), blocking was performed overnight at 4° C. The membrane was washed with 0.1% Tween20-containing TBS (TBS-0, and as the primary antibody reaction, Goat anti-cathepsin D monoclonal antibody (R&D Systems) was adjusted to 1 μg/ml using an antibody dilution solution (Can Get Signal, manufactured by TOYOBO), and the membrane was incubated for 2 hours at room temperature. After the reaction, the membrane was washed 3 times for 5 minutes, and as a secondary antibody reaction, Anti-Goat IgG-HRP (manufactured by Jackson ImmunoResearch) was adjusted to 10,000-fold dilution in TBS-t, and the membrane was incubated for 1 hour at room temperature. After the reaction, the membrane was washed for 15 minutes and 5 minutes with TBS-t, and washed with TBS for 5 minutes, after which Immunostar LD (manufactured by Wako) was added as an HRP reaction substrate, and detection was performed using a C-DiGiT blot scanner (manufactured by M&S TechnoSystems).

### [Results]

The results are shown in Table 16. The marker molecules were detected from the CD81-binding fraction of the hepatocellular carcinoma cells HAK 1A. This showed that the cathepsin D (CTSD) glycoprotein of the present invention is a type of lysosome Asp protease in the cell membrane, but at least in the case of HAK 1A cells among hepatocellular carcinoma cells, it is present as a glycoprotein contained inside or on the surfaces of CD81-positive exosomes.

1-25. (canceled)

26. A method for detecting hepatocellular carcinoma, wherein hepatocellular carcinoma is detected by in vitro detection of the hepatocellular carcinoma marker comprising an NPA lectin-binding glycoprotein in a test sample, wherein the glycoprotein is a glycoprotein chosen from among cathepsin D (CTSD), oxygen-regulated protein (HYOU1), epidermal growth factor receptor (EGFR), prosaposin (PSAP), and lysosome-associated membrane protein 2 (LAMP-2).

27. The method according to claim 26, wherein the glycoprotein is a glycoprotein that is present on the surfaces of cancer cells in hepatic tissue, or is present in the interstitium in the vicinity of the cells.

28. The method according to claim 26, wherein a glycan epitope of the NPA lectin-binding glycoprotein has at least one of the following properties (1) to (5):
(1) the glycan epitope does not include core fucose (fucose α1→6 glycan);
(2) the glycan epitope comprises a complex-type glycan having four or fewer mannoses;
(3) the glycan epitope does not include a high-mannose-type glycan having five or more mannoses;
(4) the glycan epitope comprises a complex-type glycan that does not depend on the property of binding to LCA lectin; and
(5) the glycan epitope comprises a complex-type glycan that does not depend on the property of binding to ConA lectin.

29. The method according to claim 26, wherein the in vitro detection of the hepatocellular carcinoma marker is performed by NPA staining of test cells or tissues using a labeled NPA lectin.

30. The method according to claim 26, wherein the in vitro detection of the hepatocellular carcinoma marker is performed by using a lectin array analysis method using a lectin array including NPA lectin, or by a lectin-antibody ELISA method including NPA lectin.

31. The method according to claim 30, wherein the lectin array analysis method uses a lectin array containing at least LCA lectin or ConA lectin in addition to NPA lectin.

32. The method according to claim 30, wherein the lectin-antibody ELISA method is a method for detecting the hepatocellular carcinoma marker by a sandwich method using NPA lectin and an antibody that binds to an NPA lectin-binding glycoprotein, the method being performed by immobilizing the antibody that binds to an NPA lectin-binding glycoprotein on a support, and using a lectin overlay wherein the NPA lectin-binding glycoprotein which is the hepatocellular carcinoma marker is sandwiched by a labeled NPA lectin, or using an antibody overlay wherein the NPA lectin-binding glycoprotein which is the hepatocellular carcinoma marker is sandwiched by a labeled antibody.

33. The method according to claim 32, wherein the antibody that binds to the NPA lectin-binding glycoprotein is an antibody that binds to at least one glycoprotein chosen from among CTSD, HYOU1, EGFR, PSAP, and LAMP-2.

34. The method according to claim 26, wherein the in vitro detection of the hepatocellular carcinoma marker is performed by using a blood sample containing serum components as the test sample, the method comprising a step of obtaining a fraction that is not adsorbed to the α-2,6-sialic acid-binding lectin.

35. The method according to claim 34, wherein the α-2,6-sialic acid-binding lectin is at least one lectin chosen from among SNA, SSA, TJAI and PSL1a lectin.

36. A method for determining the presence of hepatocellular carcinoma or a level of progression or malignancy of carcinoma, the method comprising:
a step of measuring, in a test sample, the reactivity of the test sample to lectins including NPA lectin, by using a lectin-antibody ELISA method or a lectin array analysis method including NPA lectin.

37. The method according to claim 36, wherein the test sample is obtained from a hepatic tissue being tested, and the method comprises:
(1) a step of preparing a discrimination formula or a calibration line corresponding to the level of progression or malignancy of hepatocellular carcinoma, by taking preliminary measurements of the reactivity of a plurality of hepatocellular carcinoma tissues and normal tissues to lectins including NPA lectin, using the lectin array analysis method or the lectin-antibody ELISA method; and
(2) a step of determining the presence of hepatocellular carcinoma or the level of progression or malignancy of carcinoma by fitting, to the discrimination formula or the calibration line, measurement values of the reactivity of the test sample to lectins including NPA lectin.

38. The method using a serum-containing sample as a test sample according to claim 36, comprising the following steps to be performed on the serum-containing test sample:
(1) a step of causing adsorption to an α-2,6-sialic acid-binding lectin immobilized on a support;
(2) a step of obtaining a fraction that is not adsorbed to the α-2,6-sialic acid-binding lectin; and
(3) a step of measuring the reactivity of the test sample to lectins including NPA lectin, using a lectin-antibody ELISA method or a lectin array analysis method including NPA lectin.

39. The method according to claim 36, comprising:
a step of measuring, in a test sample obtained from a hepatic tissue being tested, the reactivity of the test sample to lectins including NPA lectin, by using a sandwich ELISA method involving lectins including NPA lectin and an antibody that binds to at least one glycoprotein chosen from among CTSD, CFH, FBN1, FN1, HYOU1, EGFR, PSAP, and LAMP-2.

40. The method according to claim 36, comprising:
(1) a step of preparing a discrimination formula or a calibration line corresponding to the level of progression or malignancy of hepatocellular carcinoma, by taking preliminary measurements of the reactivity of a plurality of hepatocellular carcinoma tissues and normal tissues to lectins including NPA lectin, using the lectin array analysis method or the lectin-antibody ELISA method;
(2) a step of measuring the reactivity of a test sample obtained from a hepatic tissue being tested to lectins including NPA lectin, by subjecting the test sample to the lectin array or ELISA; and
(3) a step of determining the presence of hepatocellular carcinoma or the level of progression or malignancy of carcinoma by fitting measurement values of the reactivity of the test sample to lectins including NPA lectin, obtained in step (2), to the discrimination formula or the calibration line obtained in step (1).

41. The method according to claim 40, wherein the lectin array analysis method or the lectin-antibody ELISA method includes NPA lectin and LCA lectin and/or ConA lectin, and the prepared discrimination formula or calibration line further includes a discrimination formula or calibration line for LCA lectin and/or ConA lectin.

42. The method according to claim 36, comprising the following steps (1) to (4):
(1) a step of preparing a tissue section of a test sample from a hepatic tissue being tested;
(2) a step of tissue staining using fluorescent-labeled NPA lectin;
(3) a step of observing the presence or absence and the intensity of fluorescence at the cell surfaces and/or the interstitium in the vicinity thereof; and
(4) a step of determining the presence of hepatocellular carcinoma when at least a standard level of fluorescence is observed in step (3) and determining a level of progression or malignancy of the carcinoma in accordance with the intensity thereof.
